(19) United States
(12) Patent Application Publication
(10) Pub. No.: US 2025/0353899 A1
(43) Pub. Date: Nov. 20, 2025

(54) ENGINEERING MONOCLONAL ANTIBODIES TO IMPROVE STABILITY AND PRODUCTION TITER

(71) Applicant: AMGEN INC., Thousand Oaks, CA (US)

(72) Inventors: Jennitte LeAnn STEVENS, Thousand Oaks, CA (US); Deniz TEMEL, Acton, MA (US); Bram ESTES, Camarillo, CA (US); Neeraj Jagdish AGRAWAL, Natick, MA (US)

(21) Appl. No.: 19/280,627

(22) Filed: Jul. 25, 2025

Related U.S. Application Data
(63) Continuation of application No. 17/420,231, filed on Jul. 1, 2021, now abandoned.
(60) Provisional application No. 62/787,867, filed on Jan. 3, 2019.

(51) Int. Cl. C07K 16/00

(52) U.S. Cl. CPC C07K 16/00; C07K 2317/21; C07K 2317/24; C07K 2317/31; C07K 2317/94

(57) ABSTRACT

Presented herein are methods directed to engineering monoclonal antibodies and antibody variants to improve stability and their production in culture. Specifically, the monoclonal antibodies can be engineered at heavy chain residue 56 (AHo numbering) to a glycine, alanine, or serine, and/or engineered at position 80 (AHo) to be a hydrophobic residue such as alanine, isoleucine, phenylalanine, leucine, methionine, or valine.

### RELATED APPLICATIONS

The present application is a continuation of U.S. patent application Ser. No. 17/420,231, filed Jul. 1, 2021, which is a National Stage application under 35 U.S.C. § 371 of International Patent Application No. PCT/US2020/012057, having an international filing date of Jan. 2, 2020, which claims the benefit of U.S. Provisional Patent Application No. 62/787,867 filed Jan. 3, 2019.

### FIELD

The presented subject matter relaters to the field of protein engineering. Specifically, the presented subject matter relates to engineering antibodies, especially monoclonal antibodies, and variants thereof, to improve their stability and production.

### BACKGROUND

Monoclonal antibodies (mAbs) that are recombinantly produced (and active fragments thereof) are important therapeutic tools. However, as these molecules are complex, many challenges need to be met to facilitate production, storage, and therapeutic administration of these molecules.

Two challenges concern production and stability. mAbs are produced in bioreactors from engineered cells, such as Chinese Hamster Ovary (CHO) cells. However, production levels can be low and can vary between mAbs. Low production levels increase production costs, including production time, labor, and consumed resources, such as the necessary components for operating the bioreactor. Furthermore, a lack of stability impacts the “shelf-life” of mAbs. Degraded mAbs can be less potent, and fragmented mAbs can present an immunologic risk.

Therefore there is a need to improve mAb stability and production titer.

### SUMMARY

In a first aspect, provided herein are methods of increasing stability of a first antibody, comprising substituting glycine, alanine, or serine at heavy chain position 56 (AHo numbering) to create a second antibody, wherein the second antibody is more stable than the unsubstituted first antibody. For example, glycine or serine may be substituted at heavy chain position 56. For example, glycine or alanine may be substituted at heavy chain position 56. For example, glycine may be substituted at heavy chain position 56.

In a second aspect, provided herein are methods of increasing stability of a first antibody, comprising substituting a hydrophobic amino acid at heavy chain position 80 (AHo numbering) of the first antibody to create a second antibody, wherein the second antibody is more stable than the unsubstituted first antibody. Examples of hydrophobic amino acid residues include alanine, isoleucine, leucine, methionine, phenylalanine, tryptophan, tyrosine, and valine. By way of example, the hydrophobic amino acid residue can comprise or consist of alanine, isoleucine, phenylalanine, leucine, methionine, or valine. By way of example, the hydrophobic amino acid residue can comprise or consist of phenylalanine, leucine, or valine.

In a third aspect, provided herein are methods of increasing stability of a first antibody, comprising substituting alanine, phenylalanine, isoleucine, leucine, methionine, threonine, or valine at heavy chain position 80 (AHo numbering) of the first antibody to create a second antibody, wherein the second antibody is more stable than the unsubstituted first antibody. For example, phenylalanine, leucine, or valine may be substituted at heavy chain position 80. For example, isoleucine or methionine may be substituted at heavy chain position 80. For example, isoleucine may be substituted at heavy chain position 80. For example, methionine may be substituted at heavy chain position 80.

In sub-aspects of these first three aspects, the increased stability of the second antibody is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting. In some sub-aspects, the increase in titer is measured by the rate of binding to a protein A coated probe tip using an Octet Forte Bio Instrument; and/or the increased yield is measured by protein A or protein G capture; and/or the increased purity is measured by SEC of purified protein; and/or the reduction in high molecular weight species is measured by size-exclusion chromatography (SEC) and the area under the curve of each peak for each molecular weight; and/or the increased melting point temperature is measured by differential scanning fluorimetry (DSF) or differential scanning calorimetry (DSC); and/or the increased temperature of aggregation is measured by DSF; and/or the increased temperature of the onset of melting is measured by DSF.

In some sub-aspects of the first aspect, the second antibody is further substituted with a hydrophobic amino acid residue at heavy chain position 80 (AHo numbering). For example, the hydrophobic amino acid residue can comprise or consist of: alanine, isoleucine, phenylalanine, leucine, methionine, or valine. For example, the hydrophobic amino acid residue can be selected from the group consisting of: phenylalanine, leucine, and valine. In some sub-aspects of the first aspect, the second antibody is further substituted with methionine at position 80 (AHo numbering), or, alternatively, the second antibody is further substituted with isoleucine at position 80 (AHo numbering). In some sub-aspects of the first aspect, the second antibody is further substituted with alanine, phenylalanine, isoleucine, leucine, methionine, threonine, or valine at position 80 (AHo numbering). In some sub-aspects of the first aspect, the second antibody is further substituted with phenylalanine, leucine, or valine at position 80 (AHo numbering).

In some sub-aspects of the second and third aspects, the second antibody is further substituted with glycine, alanine, or serine at position 56 (AHo numbering). In some sub-aspects of the second and third aspects, the second antibody is further substituted with glycine or alanine at position 56 (AHo numbering). In some sub-aspects of the second and third aspects, the second antibody is further substituted with glycine or serine at position 56 (AHo numbering). In some sub-aspects of the second and third aspects, the second antibody is further substituted with glycine at position 56 (AHo numbering).

In these first three aspects, the first antibody is a monoclonal antibody, such as, for example, a human, or humanized, antibody. Furthermore, the first antibody is an IgG antibody, such as an IgG antibody selected from the group consisting of an IgG1, IgG2, IgG3, and IgG4 antibody. That is, the IgG antibody can be an IgG1 antibody, the IgG antibody can be an IgG2 antibody, the IgG antibody can be an IgG3 antibody, and the IgG antibody can be an IgG4 antibody.

In a fourth aspect, disclosed herein are methods of increasing stability of a first antibody variant, comprising substituting glycine, alanine, or serine at heavy chain position 56 (AHo numbering) to create a second antibody variant, wherein the second antibody variant is more stable than the unsubstituted first antibody variant. For example, glycine or serine can be substituted at heavy chain position 56. For example, glycine or alanine can be substituted at heavy chain position 56. For example, glycine can be substituted at heavy chain position 56.

In a fifth aspect, disclosed herein are methods of increasing stability of a first antibody variant, comprising substituting a hydrophobic amino acid residue (such as alanine, isoleucine, phenylalanine, leucine, methionine, or valine) at heavy chain position 80 (AHo numbering) of the first antibody variant to create a second antibody variant, wherein the second antibody variant is more stable than the unsubstituted first antibody variant. For example, the hydrophobic amino acid residue can comprise or consist of: phenylalanine, leucine, or valine. For example, the hydrophobic amino acid residue can comprise or consist of methionine or isoleucine.

In a sixth aspect, disclosed herein are methods of increasing stability of a first antibody variant, comprising substituting alanine, phenylalanine, isoleucine, leucine, methionine, threonine, or valine at heavy chain position 80 (AHo numbering) of the first antibody variant to create a second antibody variant, wherein the second antibody variant is more stable than the unsubstituted first antibody variant. For example, phenylalanine, leucine, or valine can be substituted at heavy chain position 80. For example, methionine or isoleucine can be substituted at heavy chain position 80. For example, methionine can be substituted at heavy chain position 80. For example, isoleucine can be substituted at heavy chain position 80.

In sub-aspects of these fourth, fifth, and sixth aspects, the increased stability of the second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting. In some sub-aspects, the increase in titer is measured by the rate of binding to a protein A coated probe tip using an Octet Forte Bio Instrument; and/or the increased yield is measured by protein A or protein G capture; and/or the increased purity is measured by SEC of purified protein; and/or the reduction in high molecular weight species is measured by size-exclusion chromatography (SEC) and the area under the curve of each peak for each molecular weight; and/or the increased melting point temperature is measured by differential scanning fluorimetry (DSF) or differential scanning calorimetry (DSC); and/or the increased temperature of aggregation is measured by DSF; and/or the increased temperature of the onset of melting is measured by DSF.

In some sub-aspects of the fourth aspect, the second antibody variant is further substituted with a hydrophobic amino acid residue at heavy chain position 80 (AHo numbering). For example, the hydrophobic amino acid residue can be selected from the group consisting of: alanine, isoleucine, phenylalanine, leucine, methionine, and valine. For example, the hydrophobic amino acid residue can be selected from the group consisting of: phenylalanine, leucine, and valine. In some sub-aspects of the fourth aspect, the second antibody variant is further substituted with methionine at position 80 (AHo numbering), or, alternatively, the second antibody is further substituted with isoleucine at position 80 (AHo numbering). In some sub-aspects of the fourth aspect, the second antibody variant is further substituted with alanine, phenylalanine, isoleucine, leucine, methionine, threonine, or valine at position 80 (AHo numbering). In some sub-aspects of the fourth aspect, the second antibody variant is further substituted with phenylalanine, leucine, or valine at position 80 (AHo numbering).

In some sub-aspects of the fifth and sixth aspects, the second antibody variant is further substituted with glycine, alanine, or serine at position 56 (AHo numbering). For example, the second antibody variant can be substituted with glycine or alanine at position 56. For example, the second antibody variant can be substituted with glycine or serine at position 56. For example, the second antibody variant can be substituted with glycine at position 56.

In some sub-aspects of these fourth, fifth, and sixth aspects, the first antibody variant is a multi-specific antibody, such as a bi-specific or tri-specific antibody. In some sub-aspects of these fourth, fifth, and sixth aspects, first antibody variant is an antibody fragment that can bind an antigen; the antibody fragment can be selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

Furthermore, in these fourth, fifth, and sixth aspects, the first antibody variant is a monoclonal antibody variant, such as, for example, a human, or humanized, antibody variant. Furthermore, the first antibody variant is an IgG antibody variant, such as an IgG antibody variant selected from the group consisting of an IgG1, IgG2, IgG3, and IgG4 antibody variant. That is, the IgG antibody variant can be an IgG1 antibody variant, the IgG antibody variant can be an IgG2 antibody variant, the IgG antibody variant can be an IgG3 antibody variant, and the IgG antibody variant can be an IgG4 antibody variant.

In a seventh aspect, disclosed herein are methods of increasing stability of a first antibody or first antibody variant, comprising
a. identifying the germline original amino acid sequence for the heavy chain of the first antibody or of the antibody portion of the antibody variant;
b. identifying the amino acid residues at heavy chain position 56 (AHo numbering) and heavy chain position 80 (AHo numbering) in the first antibody or of the antibody portion of the antibody variant; and
c. substituting at heavy chain positions 56 and 80 the identified residues from the germline original amino acid sequence in the first antibody or of the antibody portion of the antibody variant, thereby creating a second antibody or second antibody variant,
wherein the second antibody is more stable than the unsubstituted first antibody, or wherein the second antibody variant is more stable than the unsubstituted first antibody variant.

In a sub-aspect of this seventh aspect, the increased stability of the second antibody or second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting. In sub-aspects of this seventh aspect, the increased stability of the second antibody or second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting. In some sub-aspects, the increase in titer is measured by the rate of binding to a protein A coated probe tip using an Octet Forte Bio Instrument; and/or the increased yield is measured by protein A or protein G capture; and/or the increased purity is measured by SEC of purified protein; and/or the reduction in high molecular weight species is measured by size-exclusion chromatography (SEC) and the area under the curve of each peak for each molecular weight; and/or the increased melting point temperature is measured by differential scanning fluorimetry (DSF) or differential scanning calorimetry (DSC); and/or the increased temperature of aggregation is measured by DSF; and/or the increased temperature of the onset of melting is measured by DSF.

In this seventh aspect, the first antibody variant is a multi-specific antibody, such as a bi-specific or tri-specific antibody. In some sub-aspects of these fourth, fifth, and sixth aspects, first antibody variant is an antibody fragment that can bind an antigen; the antibody fragment can be selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

Furthermore, in this seventh aspect, the first antibody variant is a monoclonal antibody variant, such as, for example, a human, or humanized, antibody variant. Furthermore, the first antibody variant is an IgG antibody variant, such as an IgG antibody variant selected from the group consisting of an IgG1, IgG2, IgG3, and IgG4 antibody variant. That is, the IgG antibody variant can be an IgG1 antibody variant, the IgG antibody variant can be an IgG2 antibody variant, the IgG antibody variant can be an IgG3 antibody variant, and the IgG antibody variant can be an IgG4 antibody variant.

In addition, in this seventh aspect, the first antibody is a monoclonal antibody, such as, for example, a human, or humanized, antibody. Furthermore, the first antibody is an IgG antibody, such as an IgG antibody selected from the group consisting of an IgG1, IgG2, IgG3, and IgG4 antibody. That is, the IgG antibody can be an IgG1 antibody, the IgG antibody can be an IgG2 antibody, the IgG antibody can be an IgG3 antibody, and the IgG antibody can be an IgG4 antibody.

In some sub-aspects of any of the first through eighth aspects, the second antibody or second antibody variant is substituted with any one of the following pairs of residues at positions 56 and 80 of the heavy chain (AHo numbering), respectively: GF, GI, GL, GT, GV, AF, AI, AL, AV, AA, AM, SA, SI, or ST. By way of example, it is noted that for this nomenclature “GF” would refer to a “G” at heavy chain position 56 and an “F” at heavy chain position 80 (AHo numbering). In some sub-aspects of any of the first through eighth aspects, the second antibody or second antibody variant is substituted with any one of the following pairs of residues at positions 56 and 80 of the heavy chain (AHo numbering), respectively: GF, GL, GV, AF, AL, or AV. Such substitutions can have a higher titer and/or higher Tm compared to the first antibody (or first antibody variant). In some sub-aspects of any of the first through eighth aspects, the second antibody or second antibody variant is substituted with any one of the following pairs of residues at positions 56 and 80 of the heavy chain (AHo numbering), respectively: AA, AL, AM, AV, GF, GL, GT, SA, or ST. Such substitutions can have a higher titer compared to the first antibody (or first antibody variant). In some sub-aspects of any of the first through eighth aspects, the second antibody or second antibody variant is substituted with any one of the following pairs of residues at positions 56 and 80 of the heavy chain (AHo numbering), respectively: AI, AV, GI, SI, or GV. Such substitutions can have a higher Tm compared to the first antibody (or first antibody variant). In some sub-aspects of any of the first through eighth aspects, the second antibody or second antibody variant is substituted with any one of the following pairs of residues at positions 56 and 80 of the heavy chain (AHo numbering), respectively: GF, GL, GT, GV, AF, AL, AV, AA, AM, AV, SA, and ST. Such substitutions can have a higher titer compared to the first antibody (or first antibody variant). In some sub-aspects of any of the first through eighth aspects, the second antibody or second antibody variant is substituted with any one of the following pairs of residues at positions 56 and 80 of the heavy chain (AHo numbering), respectively: GF, GI, GL, GV, AF, AL, AV, AI, or SI. Such substitutions can have a higher Tm compared to the first antibody (or first antibody variant).

In an eighth aspect, provided herein are methods of making pharmaceutical compositions formulated with the second antibody or second antibody variant produced by any previous aspect.

In a ninth aspect, provided herein is an antibody or antibody variant made according to any of the first seven aspects.

In a tenth aspect, provided herein is pharmaceutical composition comprising an antibody or antibody variant made according to any of the first seven aspects.

### BRIEF DESCRIPTION OF THE DRAWINGS

FIGS. 1A-1D are a series of graphs showing various characteristics of two monoclonal antibodies: an engineered monoclonal antibody, and its parent monoclonal antibody.

FIG. 2A is a graph showing that for mAb1, alanine and glycine at HC:56 had the highest titers. HC80 residues are shown in the top row of the X-axis labels. HC56 residues are shown in the bottom row of the X-axis labels.

FIG. 2B is a graph showing that for mAb1, phenylalanine, leucine, and valine at HC:80 had the highest titers. HC56 residues are shown in the top row of the X-axis labels. HC80 residues are shown in the bottom row of the X-axis labels.

FIG. 3A is a graph showing that for mAb1, the HC56 and HC80 variants with high titers also had high Tm's. HC80 residues are shown in the top row of the X-axis labels. HC56 residues are shown in the bottom row of the X-axis labels.

FIG. 3B is a graph showing that for mAb1, molecules with phenylalanine, leucine, or valine at HC80 had Tm's above 65° C. HC56 residues are shown in the top row of the X-axis labels. HC80 residues are shown in the bottom row of the X-axis labels.

FIG. 4 is a graph showing that for mAb1, most high molecular weight (HMW) levels were below 5%, as determined by SEC. HC80 residues are shown in the top row of the X-axis labels. HC56 residues are shown in the bottom row of the X-axis labels.

FIG. 5 is a graph showing that mAb2 expresses well with residues such as hydrophobic residues at HC56 and HC80. HC80 residues are shown in the top row of the X-axis labels. HC56 residues are shown in the bottom row of the X-axis labels.

FIG. 6 is a graph showing that for substitutions at HC56 and HC80 of mAb2, Tm correlates with titer. HC80 residues are shown in the top row of the X-axis labels. HC56 residues are shown in the bottom row of the X-axis labels.

FIG. 7 is a graph showing high molecular weight species for substitutions at HC56 and HC80 of mAb2. HC80 residues are shown in the top row of the X-axis labels. HC56 residues are shown in the bottom row of the X-axis labels.

### DETAILED DESCRIPTION

Surprisingly, when the antibody heavy chain residue 56 (AHo numbering; residue 49 in Kabat numbering) of mAbs is modified to be a glycine, the antibody has a higher titer in culture/during production and a higher Tm than molecules with the frequently observed alanine residue at that position. This effect has been observed in comparisons across a number of mAbs and germlines and is case-independent of which residue is germline. This observation is in contrast to the published work by Mason et al. (Mason et al 2012), who reports that alanine at residue 56 (AHo numbering) improves expression titer of IgG4 mAbs. In addition, titers are heavily impacted using methionine versus isoleucine residues at heavy chain 80 position (AHo numbering) in a molecule-dependent manner.

### Definitions

The AHo numbering scheme is a structure-based numbering scheme, which introduces gaps in the CDR regions to minimize deviation from the average structure of the aligned domains (Honegger & Pluckthun 2001). In the AHo numbering scheme, structurally equivalent positions in different antibodies will have the same residue number.

“Antibody” or “immunoglobulin” refers to a tetrameric glycoprotein that consists of two heavy chains and two light chains, each comprising a variable domain (V) and a constant domain (C). “Heavy chains” and “light chains” refer to substantially full-length canonical immunoglobulin light and heavy chains; the variable domains (VL and VC) of the heavy and light chains constitute the V region of the antibody and contributes to antigen binding and specificity. “Antibody” includes monoclonal antibodies, polyclonal antibodies, chimeric antibodies, human antibodies, and humanized antibodies. Light chains can be classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including IgG1, IgG2, IgG3, and IgG4. IgM has subclasses including IgM1 and IgM2. IgA is similarly subdivided into subclasses including IgA1 and IgA2. Within full-length light and heavy chains, typically, the variable and constant regions are joined by a “J” region of about 12 or more amino acids, with the heavy chain also including a “D” region of about 10 more amino acids. The variable regions of each light/heavy chain pair typically form the antigen binding site. “Monoclonal antibody” refers to an antibody obtained from a population of substantially homogeneous antibodies, i.e., the individual antibodies comprising the population are identical except for possible naturally occurring mutations that may be present in minor amounts.

“Antibody variants” include antibody fragments and antibody-like proteins with changes to structure of canonical tetrameric antibodies. Typical antibody variants include V regions with a change to the constant regions, or, alternatively, adding V regions to constant regions, optionally in a non-canonical way. Examples include multi-specific antibodies (e.g., bispecific antibodies, trispecific antibodies), antibody fragments that can bind an antigen (e.g., Fab′, F′(ab)2, Fv, single chain antibodies, diabodies), biparatopic and recombinant peptides comprising the forgoing as long as they exhibit the desired biological activity.

Multi-specific antibodies target more than one antigen or epitope. For example, a “bispecific,” “dual-specific”, or “bifunctional” antibody is a hybrid antibody that has two different antigen binding sites. Bispecific antibodies can be produced by a variety of methods including fusing hybridomas or linking Fab′ fragments (Kostelny et al 1992, Songsivilai & Lachmann 1990) (Kostelny et al 1992, Songsivilai & Lachmann 1990, Wu & Demarest 2018). The two binding sites of a bispecific antibody each bind to a different epitope. Likewise, trispecific antibodies have three binding sites and bind three epitopes. Several methods of making trispecific antibodies are known and are being further developed (Wu & Demarest 2018, Wu et al 2018)

“Antibody fragments” include antigen-binding portions of the antibody including, for example, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired binding activity.

### Overview of Methods

The disclosed methods include steps of identifying the residues at positions 56 and 80 (AHo numbering) of the heavy chain or the germline progenitor amino acid residue at these positions in an antibody (or modified antibody), changing (mutating) the residue at position 56 to glycine, alanine, or serine and/or residue 80 to a hydrophobic residue (such as methionine or isoleucine) or to any of alanine, phenylalanine, isoleucine, leucine, methionine, threonine, or valine, and assessing stability of the modified antibody, using any of a variety of techniques that measure different characteristics of the antibody. In some embodiments, the residue at heavy chain position 56 is changed to glycine.

The following discussion is applicable not only to antibodies, including IgG's (IgG1, IgG2, IgG3, and IgG4), but also to antibody variants, which are described in the “Definitions” section, above.

Identifying Residues at Position 56 and/or 80 (AHo Numbering)

In a first step, the amino acid residue at 56 and/or 80 (Aho numbering) in the heavy chain of the Ab is identified. If position 56 is already a glycine, then no further identification is necessary, as the Ab needs no further engineering at this position. In most cases, the antibody polynucleotide sequences are cloned and sequenced, and the sequence then translated to the amino acid sequence. Alternatively, relevant amino acid sequences from an antibody or region thereof (e.g., variable region) can be determined by direct protein sequencing. In some embodiments, if position 56 is already a glycine, alanine, or serine, then no further identification is necessary, as the Ab needs no further engineering at this position. In some embodiments, if position 56 is already a glycine or serine, then no further identification is necessary, as the Ab needs no further engineering at this position. In some embodiments, if position 56 is already a glycine or alanine, then no further identification is necessary, as the Ab needs no further engineering at this position. In some embodiments, if position 56 is already a glycine, then no further identification is necessary, as the Ab needs no further engineering at this position.

Alternatively, genomic or cDNA that encode the monoclonal antibody of interest or binding fragments thereof can be isolated and sequenced from cells producing such antibodies using conventional procedures (e.g., by using oligonucleotide probes that are capable of binding specifically to genes encoding the heavy and light chains of the monoclonal antibodies).

DNA sequencing can be performed by any technique known in the art, such as described by Sanger et al (Sanger et al 1977) or high-throughput sequencing methods, such as pyrosequencing (Margulies et al 2005, Nyren & Lundin 1985, Ronaghi et al 1998), sequencing by synthesis (Bentley et al 2008), ion semiconductor (Rothberg et al 2011), single-molecule real-time sequencing (Eid et al 2009), sequencing by oligo ligation detection (SOLiD) (Valouev et al 2008), and nanopore sequencing (discussed in Branton et al. (Branton et al 2008)).

Once the polynucleotide sequence is obtained, the open reading frames are determined, and the amino acid sequence deduced according to the genetic code. AHo numbering is applied to determine position 56 and/or 80, and the amino acid residue determine.

Direct protein sequencing of the antibody is also possible. Protein sequencing methods include, for example, by using mass spectrometry, as well as Edman degradation approaches using a protein sequencer. In the case of Edman degradation, since the target antibody is likely longer than 50-70 amino acids, the antibody can be digested with an endopeptidase (such as trypsin or pepsin) or chemically, using cyanogen bromide, BNPS-skatolet, formic acid, or chloramine T. The target size of the fragments for Edman degradation is 50-70 amino acids. Once the antibody has been fragmented, then the peptides can be analyzed in an automated way using a protein sequenator which performs the Edman degradation reaction and reads each released amino acid by a detection method, such as high-pressure liquid chromatography (HPLC). For mass spectrometry, the antibody can be fragmented with a protease (commonly trypsin), the fragments separated by liquid chromatography (LC) and the fragments analyzed with a mass spectrometer, using de novo peptide sequencing algorithms; this approach is discussed by Medzihradszky and Chalkley (Medzihradszky & Chalkley 2015).

As described above, once the sequence is determined and AHo numbering applied, the amino acid at position 56 and/or 80 can be determined.

### Engineering Residue(s) to Target Residues

Regardless of the method chosen to identify the residue at AHo position 56 and/or 80 of antibody heavy chains, a decision is made to whether to mutate the residue at the polynucleotide level. In some embodiments, if the residue at position 56 is not glycine, then the residue is a candidate for being changed. By way of example, in the most common scenario, the residue at position 56 when not a glycine is an alanine. Thus an A56G mutation can be made. Likewise, for position 80, if the residue is not a hydrophobic residue (or is not any of alanine, phenylalanine, isoleucine, leucine, methionine, threonine, or valine), the residue is a candidate for being changed to a hydrophobic residue (or to any of alanine, phenylalanine, isoleucine, leucine, methionine, threonine, or valine, for example methionine). By way of example, in some embodiments, in the case of this position (80), the residue is not methionine, the residue is a candidate for being changed to methionine. By way of example, in some embodiments, in the case of this position (80), if the residue is not an isoleucine, then the residue is a candidate for being changed to isoleucine. In the case of this position (80), even if the residue is a hydrophobic residue, such as methionine or isoleucine, the position is still a candidate for change to a different hydrophobic residue (such as isoleucine or methionine, respectively, because either of these amino acids (Met, lie) can increase expression and stability in culture). In some embodiments, in the case of this position (80), the residue can be changed to any of alanine, phenylalanine, isoleucine, leucine, methionine, threonine, or valine, or to a hydrophobic residue (such as alanine, phenylalanine, isoleucine, leucine, or methionine), even if that residue is already a different one of alanine, phenylalanine, isoleucine, leucine, methionine, threonine, or valine, or a different hydrophobic residue. If the residue at position 56 is not glycine, then the residue may also be a candidate for being changed, for example to a glycine. Examples of hydrophobic amino acid residues include alanine, isoleucine, leucine, methionine, phenylalanine, tryptophan, tyrosine, and valine. In some embodiments, for any method describe herein, in the case of heavy chain position 80, the hydrophobic amino acid residue is selected from the group consisting of: phenylalanine, leucine, and valine.

Any known method can be used to modify the polynucleotide encoding the antibody of interest. After determining the amino acid residues at position 56 and/or 80, the nucleic acid sequence is modified so that glycine (or alanine or serine) is encoded at position 56, and/or alanine, phenylalanine, isoleucine, leucine, methionine, threonine, or valine (or a hydrophobic amino acid residue, or methionine or isoleucine) is encoded at position 80. To make this change, the codon encoding the amino acid at position 56 and/or 80 is identified, and a mutation, or mutations, selected according to Table 1, which shows the genetic code.

TABLE 1
The genetic code

| | U | C | A | G | |
|---|---|---|---|---|---|
| U | UUU Phenylalanine (Phe) | UCU Serine (Ser) | UAU Tyrosine (Tyr) | UGU Cysteine (Cys) | U |
| | UUC Phe | UCC Ser | UAC Tyr | UGC Cys | C |
| | UUA Leucine (Leu) | UCA Ser | UAA STOP | UGA STOP | A |
| | UUG Leu | UCG Ser | UAG STOP | UGG Tryptophan (Trp) | G |
| C | CUU Leucine (Leu) | CCU Proline (Pro) | CAU Histidine (His) | CGU Arginine (Arg) | U |
| | CUC Leu | CCC Pro | CAC His | CGC Arg | C |
| | CUA Leu | CCA Pro | CAA Glutamine (Gln) | CGA Arg | A |
| | CUG Leu | CCG Pro | CAG Gln | CGG Arg | G |
| A | AUU Isoleucine (Ile) | ACU Threonine (Thr) | AAU Asparagine (Asn) | AGU Serine (Ser) | U |
| | AUC Ile | ACC Thr | AAC Asn | AGC Ser | C |
| | AUA Ile | ACA Thr | AAA Lysine (Lys) | AGA Arginine (Arg) | A |
| | AUG Methionine (Met) or START | ACG Thr | AAG Lys | AGG Arg | G |
| G | GUU Valine Val | GCU Alanine (Ala) | GAU Aspartic acid (Asp) | GGU Glycine (Gly) | U |
| | GUC (Val) | GCC Ala | GAC Asp | GGC Gly | C |
| | GUA Val | GCA Ala | GAA Glutamic acid (Glu) | GGA Gly | A |
| | GUG Val | GCG Ala | GAG Glu | GGG Gly | G |

Most amino acids are encoded by more than one codon as shown in Table 1. For instance, alanine is encoded by four codons: GCU, GCC, GCA, and GCG; however, only Trip and Met are each encoded by a single codon (TGG and ATG, respectively). When selecting a mutation or more than one mutation, considerations regarding, for example, codon bias, can be accounted for (Quax et al 2015).

Standard techniques can be used to introduce mutations in the nucleotide sequence encoding an antibody of the present disclosure, including site-directed mutagenesis and polymerase chain reaction (PCR)-mediated mutagenesis which result in the targeted amino acid substitutions. Commercial kits are also available that can accomplish introducing mutations into nucleic acids, such as GeneArt™ systems and Phusion kits (ThermoFisher Scientific; Waltham, MA); Q5® site-directed mutagenesis kit (New England Biol-abs; Ipswich, MA); and customizable kits from Civic Bioscience (Montreal, Canada).

Alternatively, a polynucleotide fragment can be synthesized using art-known techniques and substituted within a polynucleotide comprising the full coding sequence. In some cases, the entire coding sequence with the targeted mutation(s) is synthesized.

In any case, amino acid mutation method is not particularly limited if it can effectively realize the site mutation.

Cell Selection and Transfection with Engineered Polynucleotides

Recombinant DNA methods for producing antibodies are well-known. DNA encoding the antibodies, for example, DNA encoding a VH domain, a VL domain, a single chain variable fragment (scFv), or fragments and combinations thereof (target polynucleotides), can be inserted into a suitable expression vector, which can then be transfected into a suitable host cell, such as Escherichia coli cells, COS cells, Chinese Hamster Ovary (CHO) cells, or myeloma cells that do not otherwise produce an antibody, to obtain the desired antibodies.

Suitable expression vectors are known in the art, containing, for example a polynucleotide that encodes the target polypeptide linked to a promoter. Such vectors can include the nucleotide sequence encoding the constant region of the antibody molecule, and the variable domain of the antibody can be cloned into such a vector for expression of the heavy chain, the entire light chain, or both the entire heavy and light chains (or fragments thereof). The expression vector can be transferred to a host cell by conventional techniques, and the transfected cells can be cultured to produce the antibodies.

Any cell line that can express, or is engineered to express, functional antibody or antibody fragments, can be used. For example, suitable mammalian cell lines include immortalized cell lines available from the American Type Culture Collection (Manassas, VA), including Chine Hamster Ovary (CH)) cells, HeLa cells, baby hamster kidney (BHK) cells, monkey kidney cells (COS), human hepatocellular carcinoma cells (e.g., Hep G2), and human epithelial kidney 293 cells. Furthermore, cell lines or host systems can be chosen to ensure correct modification and processing of antibodies. Eukaryotic host cells that possess the cellular machinery for proper processing of the primary transcript, glycosylation, and phosphorylation of the gene product can be used. These include CHO, VERY, BHK, Hela, COS, MDCK, 293, 3T3, W138, BT483, Hs578T, HTB2, BT20 and T47D, NS0 (a murine myeloma cell line that does not endogenously produce any functional immunoglobulin chains), SP20, CRL7030 and HsS78Bst cells. Human cell lines developed by immortalizing human lymphocytes can also be used. The human cell line PER.C6® (Janssen; Titusville, NJ) can be used to recombinantly produce monoclonal antibodies. Examples of non-mammalian cells that can also be used include insect cells (e.g., Sf21/Sf9, Trichoplusia ni Bti-Tn5bl-4), or yeast cells (e.g., Saccharomyces (such as S. cerevisiae, Pichia, etc.), plant cells, or chicken cells.

Antibodies can be stably expressed in a cell line using conventional methods. Stable expression can be used for long-term, high-yield production of recombinant proteins. For stable expression, host cells can be transformed with an appropriately engineered vector that includes expression control elements (e.g., promoter, enhancer, transcription terminators, polyadenylation sites, etc.), and a selectable marker gene. Methods for producing stable cell lines with a high yield are known in the art and reagents are available commercially. Transient expression can also be accomplished using conventional methods.

A cell line expressing an antibody can be maintained in cell culture medium and under culture conditions that result in the expression and production of the antibodies. Cell culture media can be based on commercially available media formulations, including, for example, DMEM or Ham's F12. In addition, the cell culture media can be modified to support increases in both cell growth and biologic protein expression. Of course, cell culture medium can be optimized for a specific cell culture, including cell culture growth medium which is formulated to promote cellular growth or cell culture production medium which is formulated to promote recombinant protein production.

Many cell culture media and cell culture nutrients and supplements are known. For example, suitable basal media include Dulbecco's Modified Eagle's Medium (DMEM), DME/F12, Minimal Essential Medium (MEM), Basal Medium Eagle (BME), RPMI 1640, F-10, F-12, a-Minimal Essential Medium (a-MEM), Glasgow's Minimal Essential Medium (G-MEM), PF CHO, and Iscove's Modified Dulbecco's Medium. Other examples of basal media that can be used include BME Basal Medium, Dulbecco's Modified Eagle Medium.

The basal medium may be serum-free, meaning that the medium contains no serum (e.g., fetal bovine serum (FBS)) or animal protein-free media or chemically-defined media. The basal medium can be modified in order to remove certain non-nutritional components found in basal media, such as various inorganic and organic buffers, surfactant(s), and sodium chloride. The cell culture medium can contain a basal cell medium (modified or not), and at least one of the following: iron source, recombinant growth factor; buffer; surfactant; osmolarity regulator; energy source; and non-animal hydrolysates. In addition, the modified basal cell medium can optionally contain amino acids, vitamins, or a combination of both amino acids and vitamins. A modified basal medium can further contain glutamine, e.g., L-glutamine, and/or methotrexate.

### Purification

Once an antibody has been produced, it can be purified by conventional methods, for example, by chromatography (e.g., ion exchange, affinity, particularly by affinity for the specific antigens, Protein A, Protein G, or sizing column chromatography), centrifugation, differential solubility, or by any other standard technique for the purification of proteins. Further, the antibodies can be fused to heterologous polypeptide sequences (“tags”) to facilitate purification.

### Assessing Stability

### Titer in Culture

Supernatants from cell cultures expressing the engineered polynucleotides can be assayed, for example, using Octet® platform instruments (Pall ForteBio; Fremont, CA). The Octet® platform provides biosensors for a number of different analytes, including biosensors for Anti-Human IgG Quantitation (AHQ), Anti-Murine IgG Quantitation (AMQ), Anti-FLAG (FLG), Protein A (ProA), Protein G (ProG), Protein L (ProL), Anti-Penta-His (HIS), Streptavidin (SA), Anti-Human Fab-cH1 (FAb), Anti-GST (GST), and Ni-NTA (NTA). Other options include using traditional ELISA formats, as well as HPLC and radioimmunoassay (RIA).

### Melting Temperature (Differential Scanning Fluorimetry (DSF) and Differential Scanning Calorimetry (DSC))

Thermal stability by differential scanning fluorimetry (DSF) (also known as Protein Thermal Shift Assay; (Lo et al 2004, Pantoliano et al 2001, Semisotnov et al 1991)) can be conveniently used to determining a protein's melting temperature, Tm. DSF takes advantage of fluorescent dyes that preferentially bind to unfolded (denatured) proteins. A real-time polymerase chain reaction (PCR) instrument is often used to monitor thermally induced protein denaturation by measuring changes in fluorescence of the dye. Examples of useful dyes include SYPRO® Orange (Thermo Fisher Scientific; Waltham, MA), 8-Anilinonaphthalene-1-sulfonic acid (ANS), N-[4-(7-diethylamino-4-methyl-3-coumarinyl)phenyl]maleimide (CPM), and 4-(dicyanovinyl)julolidine (DCVJ). The method of Lo et al. is often used, taking advantage of commonly used RE-PCR instruments and SYPRO® Orange (Lo et al 2004).

In general, the dye and protein(s) being analyzed are mixed, a melt curve is determined, and the Tm is calculated from the melt curve.

In addition to DSF, any known technique to determine the Tm of a protein can be used. For example, techniques that take advantage of intrinsic fluorescence signals (such as from tryptophan) can be used, as well as different modes of monitoring protein folding/unfolding, such as light scattering. Other techniques include fast parallel proteolysis (Minde et al 2012) and cellular thermal shift assay (Jafari et al 2014).

Differential scanning calorimetry (DSC) can also be used to determine the Tm of a protein. (Makhatadze 1998), although because DSC can also provide information on the modes of unfolding, multiple Tm values may be obtained.

In general, the spectrum of the subject protein is determined using a spectrophotometer to quantify the protein (or alternatively, the protein is quantified using a different method). Then, the protein is subjected to the DSC program of the spectrophotometer.

### Yield (as Measured by Antibody Captured by Protein A)

A generic process for antibody purification from clarified cell culture supernatant contains a capture step with protein A affinity chromatography; followed by a combination of anion and cation exchange chromatography (Fahrner et al 2001, Kelley 2009)

Protein quantification can be performed using any method known in the art. These include UV-Vis spectroscopy at 280 nm (A280); based on the absorbance of tryptophan and tyrosine residues (or alternatively absorbance at 205 nm (A205), detecting protein backbones; the Bradford assay (usually based on the use and absorbance of Coomassie Brilliant Blue G-250 dye); Biuret Test-derived assays, such as Lowry and Bicinchoninic acid (BCA) assays; amino acid analysis (depending on the directed detection of modified amino acids); gel electrophoresis (as observed by gel band intensity), or dye-labeling the protein (thus correlating detection of the dye signal to protein quantity), examples of dyes include fuorescamine and Amido black 10B. Additionally, HPLC and LC/MS methods can also be used.

To conserve sample and facilitate measurements, a NanoDrop spectrophotometer can be used, such as available from Thermo Fisher Scientific (Wilmington, DE). This device facilitates several approaches to quantify proteins.

### High Molecular Weight (HMW) and Main Peak (MP) Species as Determined by Size-Exclusion Chromatography (SEC)

A generic process for SEC to compare species of protein A eluted material is used, in which the protein is run through a column of porous beads of dextran polymers to separate species based on size. The percent HMW versus MP is determined by measuring the area under the curves of the SEC peaks.

### Aggregation (T Aggregation) and Onset of Melting (T Onset of Melting or T Onset Melting)

T aggregation is measured by Differential Scanning Fluoremetry (DSF) and measures the temperature at which 30 nm aggregate particles form using an excitation wavelength of 300 nm and emissions of 350/330 nm.

T onset of melting is measured by DSF and measures the Fluorescence at 350/330 nm. T onset is the temperature at which the first derivative of 350/330 nm emissions rises above the base level, representative of when folded protein begins to unfold

### Pharmaceutical Composition Formulation and Components

The antibodies and antibody variants made according to the methods disclosed herein can be formulated into pharmaceutical compositions, suitable for administration to a patient.

Acceptable pharmaceutical components preferably are nontoxic to patients at the dosages and concentrations used. Pharmaceutical compositions can comprise agents for modifying, maintaining or preserving, for example, the pH, osmolarity, viscosity, clarity, color, isotonicity, odor, sterility, stability, rate of dissolution or release, adsorption or penetration of the composition.

In general, excipients can be classified on the basis of the mechanisms by which they stabilize proteins against various chemical and physical stresses. Some excipients alleviate the effects of a specific stress or regulate a particular susceptibility of a specific polypeptide. Other excipients more generally affect the physical and covalent stabilities of proteins.

Common excipients of liquid and lyophilized protein formulations are shown in Table 2 (see also (Kamerzell et al 2011)).

TABLE 2
Examples of excipient components for polypeptides formulations

| Component | Function | Examples |
|---|---|---|
| Buffers | Maintaining solution pH | Citrate, Succinate, Acetate, |
| | Mediating buffer-ion specific interactions | Glutamate, Aspartate, Histidine, |
| | with polypeptides | Phosphate, Tris, Glycine |
| Sugars and | Stabilizing polypeptides | Sucrose, Trehalose, Sorbitol, |
| carbohydrates | Tonicifying agents | Mannitol, Glucose, Lactose, |
| | | Cyclodextrin derivatives |
| | Acting as carriers for inhaled drugs (e.g., | |
| | lactose) | |
| | Providing dextrose solutions during IV | |
| | administration | |
| Stabilizers and | Enhancing product elegance and preventing | Mannitol, Glycine |
| bulking agents | blowout | |
| | Providing structural strength to a lyo cake | |
| Osmolytes | Stabilizing against environmental stress | Sucrose, Trehalose, Sorbitol, |
| | (temperature, dehydration) | Glycine, Proline, Glutamate, |
| | | Glycerol, Urea |
| Amino acids | Mediating specific interactions with | Histidine, Arginine, Glycine, |
| | polypeptides | Proline, Lysine, Methionine, |
| | Providing antioxidant activity (e.g., His, Met) | Amino acid mixtures (e.g., |
| | Buffering, tonicifying | Glu/Arg) |
| Polypeptides and | Acting as competitive inhibitors of | HSA, PVA, PVP, PLGA, PEG, |
| polymers | polypeptide adsorption | Gelatin, Dextran, Hydroxyethyl |
| | Providing bulking agents for lyophilization | starch, HEC, CMC |
| | Acting as drug delivery vehicles | |
| Anti-oxidants | Preventing oxidative polypeptides damage | Reducing agents, Oxygen |
| | Metal ion binders (if a metal is included as a | scavengers, Free radical |
| | cofactor or is required for protease | scavengers, Chelating agents (e.g., |
| | activity) | EDTA, EGTA, DTPA), Ethanol |
| | Free radical scavengers | |
| Metal ions | Polypeptides cofactors | Magnesium, Zinc |
| | Coordination complexes (suspensions) | |
| Specific ligands | Stabilizers of native conformation against | Metals, Ligands, Amino acids, |
| | stress-induced unfolding | Polyanions |
| | Providing conformation flexibility | |
| Surfactants | Acting as competitive inhibitors of | Polysorbate 20, Polysorbate 80, |
| | polypeptides adsorption | Poloxamer 188, Anionic |
| | Acting as competitive inhibitor of | surfactants (e.g., sulfonates and |
| | polypeptides surface denaturation | sulfosuccinates), Cationic |
| | Providing liposomes as drug delivery | surfactants, Zwitterionic |
| | vehicles | surfactants |
| | Inhibiting aggregation during lyophilization | |
| | Acting as reducer of reconstitution times of | |
| | lyophilized products | |
| Salts | Tonicifying agents | NaCl, KCl, NaSO₄ |
| | Stabilizing or destabilizing agents for | |
| | polypeptides, especially anions | |
| Preservatives | Protecting against microbial growth | Benzyl alcohol, M-cresol, Phenol |

Other excipients are known in the art (e.g., see (Powell et al 1998). Those skilled in the art can determine what amount or range of excipient can be included in any particular formulation to achieve a biopharmaceutical composition that promotes retention in stability of the biopharmaceutical. For example, the amount and type of a salt to be included in a biopharmaceutical composition can be selected based on to the desired osmolality (i.e., isotonic, hypotonic or hypertonic) of the final solution as well as the amounts and osmolality of other components to be included in the formulation.

### EMBODIMENTS

The following embodiments are presented as examples of the methods disclosed herein are non-limiting. The Examples section follows this embodiments section.

Embodiment 1. A method of increasing stability of a first antibody, comprising substituting glycine, alanine, or serine at heavy chain position 56 (AHo numbering) to create a second antibody, wherein the second antibody is more stable than the unsubstituted first antibody. For example, glycine may be substituted at heavy chain position 56. For example, glycine or alanine may be substituted at heavy chain position 56. For example, glycine or serine may be substituted at position 56.

Embodiment 2. The method of Embodiment 1, wherein the glycine is substituted at heavy chain position 56.

Embodiment 3. The method of any one of Embodiments 1-2, wherein the second antibody is further substituted with a hydrophobic amino acid residue at heavy chain position 80 (AHo numbering).

Embodiment 4. The method of Embodiment 3, wherein the hydrophobic amino acid residue is selected from the group consisting of: alanine, isoleucine, phenylalanine, leucine, methionine, and valine.

Embodiment 5. The method of Embodiment 3, wherein the hydrophobic amino acid residue is selected from the group consisting of: phenylalanine, leucine, and valine.

Embodiment 6. The method of any one of Embodiments 1-2, wherein the second antibody is further substituted with methionine at position 80 (AHo numbering).

Embodiment 7. The method of any one of Embodiments 1-2, wherein the second antibody is further substituted with isoleucine at position 80 (AHo numbering).

Embodiment 8. A method of increasing stability of a first antibody, comprising substituting a hydrophobic amino acid residue at heavy chain position 80 (AHo numbering) of the first antibody to create a second antibody, wherein the second antibody is more stable than the unsubstituted first antibody.

Embodiment 9. The method of Embodiment 8, wherein the hydrophobic amino acid residue is selected from the group consisting of: alanine, isoleucine, phenylalanine, leucine, methionine, and valine.

Embodiment 10. The method of Embodiment 8, wherein the hydrophobic amino acid residue is selected from the group consisting of: phenylalanine, leucine, and valine.

Embodiment 11. A method of increasing stability of a first antibody, comprising substituting alanine, phenylalanine, isoleucine, leucine, methionine, threonine, or valine at heavy chain position 80 (AHo numbering) of the first antibody to create a second antibody, wherein the second antibody is more stable than the unsubstituted first antibody.

Embodiment 12. The method of Embodiment 11, wherein the methionine is substituted at heavy chain position 80 of the first antibody.

Embodiment 13. The method of Embodiment 11, wherein the isoleucine is substituted at heavy chain position 80 of the first antibody.

Embodiment 14. The method of any one of Embodiments 8-13, wherein the second antibody is further substituted with alanine, glycine, or serine at heavy chain position 56 (AHo numbering).

Embodiment 15. The method of any one of Embodiments 8-13, wherein the second antibody is further substituted with alanine or glycine at heavy chain position 56 (AHo numbering).

Embodiment 16. The method of any one of Embodiments 8-13, wherein the second antibody is further substituted with glycine at heavy chain position 56 (AHo numbering)

Embodiment 17. The method of any of Embodiments 1-16, wherein the increased stability of the second antibody is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

Embodiment 18. The method of Embodiment 17, wherein the increase in titer is measured by the rate of binding to a protein A coated probe tip using an Octet Forte Bio Instrument.

Embodiment 19. The method of Embodiment 17, wherein the increased yield is measured by protein A or protein G capture.

Embodiment 20. The method of Embodiment 17, wherein the increased purity is measured by size-exclusion chromatography (SEC) of the purified antibodies.

Embodiment 21. The method of Embodiment 17, wherein the reduction in high molecular weight species is measured by size-exclusion chromatography (SEC) and area under the curve for each peak at each molecular weight.

Embodiment 22. The method of Embodiment 17, wherein the increased melting point temperature is measured by differential scanning fluorimetry (DSF) or differential scanning calorimetry (DSC).

Embodiment 23. The method of Embodiment 17, wherein the increased temperature of aggregation is measured by DSF.

Embodiment 24. The method of Embodiment 17, wherein the increased temperature of the onset of melting is measured by DSF.

Embodiment 25. The method of any preceding Embodiment, wherein the first antibody is a monoclonal antibody.

Embodiment 26. The method of any preceding Embodiment, wherein the first antibody is a human monoclonal antibody or a humanized monoclonal antibody.

Embodiment 27. The method of any preceding Embodiment, wherein the first antibody is an IgG antibody.

Embodiment 28. The method of Embodiment 27, wherein the IgG antibody is selected from the group consisting of an IgG1, IgG2, IgG3, and IgG4 antibody.

Embodiment 29. The method of Embodiment 27, wherein the IgG antibody is an IgG1 antibody.

Embodiment 30. The method of Embodiment 27, wherein the IgG antibody is an IgG2 antibody.

Embodiment 31. The method of Embodiment 27, wherein the IgG antibody is an IgG3 antibody.

Embodiment 32. The method of Embodiment 27, wherein the IgG antibody is an IgG4 antibody.

Embodiment 33. A method of increasing stability of a first antibody variant, comprising substituting glycine, alanine, or serine at heavy chain position 56 (AHo numbering) to create a second antibody variant, wherein the second antibody variant is more stable than the unsubstituted first antibody variant.

Embodiment 34. The method of Embodiment 33, wherein the glycine is substituted at heavy chain position 56.

Embodiment 35. The method of any one of Embodiments 33-34, wherein the second antibody variant is further substituted with a hydrophobic amino acid residue at heavy chain position 80 (AHo numbering).

Embodiment 36. The method of Embodiment 35, wherein the hydrophobic amino acid residue is selected from the group consisting of: alanine, isoleucine, phenylalanine, leucine, methionine, and valine.

Embodiment 37. The method of Embodiment 35, wherein the hydrophobic amino acid residue is selected from the group consisting of: phenylalanine, leucine, and valine.

Embodiment 38. A method of increasing stability of a first antibody variant, comprising substituting a hydrophobic amino acid residue at heavy chain position 80 (AHo numbering) of the first antibody variant to create a second antibody variant, wherein the second antibody variant is more stable than the unsubstituted first antibody variant.

Embodiment 39. The method of Embodiment 38, wherein the hydrophobic amino acid residue is selected from the group consisting of: alanine, isoleucine, phenylalanine, leucine, methionine, and valine.

Embodiment 40. The method of Embodiment 38, wherein the hydrophobic amino acid residue is selected from the group consisting of: phenylalanine, leucine, and valine.

Embodiment 41. A method of increasing stability of a first antibody variant, comprising substituting alanine, phenylalanine, isoleucine, leucine, methionine, threonine, or valine at heavy chain position 80 (AHo numbering) of the first antibody variant to create a second antibody variant, wherein the second antibody variant is more stable than the unsubstituted first antibody variant.

Embodiment 42. The method of Embodiment 41, wherein the methionine is substituted at heavy chain position 80 of the first antibody variant.

Embodiment 43. The method of Embodiment 41, wherein the isoleucine is substituted at heavy chain position 80 of the first antibody variant.

Embodiment 44. The method of any one of Embodiments 38-43, wherein the second antibody variant is further substituted with alanine, glycine, or serine at heavy chain position 56 (AHo numbering).

Embodiment 45. The method of any one of Embodiments 38-43, wherein the second antibody variant is further substituted with alanine or glycine at heavy chain position 56 (AHo numbering).

Embodiment 46. The method of any one of Embodiments 38-43, wherein the second antibody variant is further substituted with glycine at heavy chain position 56 (AHo numbering) Embodiment 47. The method of any of Embodiments 33-46, wherein the increased stability of the second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

Embodiment 48. The method of Embodiment 47, wherein the increase in titer is measured by the rate of binding to a protein A coated probe tip using an Octet Forte Bio Instrument.

Embodiment 49. The method of Embodiment 47, wherein the increased yield is measured by protein A or protein G capture.

Embodiment 50. The method of Embodiment 47, wherein the increased purity is measured by SEC of the purified antibodies.

Embodiment 51. The method of Embodiment 47, wherein the reduction in high molecular weight species is measured by SEC and area under the curve for each peak at each molecular weight.

Embodiment 52. The method of Embodiment 47, wherein the increased melting point temperature is measured by DSF or DSC.

Embodiment 53. The method of Embodiment 47, wherein the increased temperature of aggregation is measured by DSF.

Embodiment 54. The method of Embodiment 47, wherein the increased temperature of the onset of melting is measured by DSF.

Embodiment 55. The method of any of Embodiments 33-54, wherein the first antibody variant is a multi-specific antibody.

Embodiment 56. The method of Embodiment 55, wherein the multi-specific antibody is a bispecific antibody or trispecific antibody.

Embodiment 57. The method of any of Embodiments 33-56, wherein the first antibody variant is an antibody fragment that can bind an antigen.

Embodiment 58. The method of Embodiment 57, wherein the antibody fragment is selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

Embodiment 59. The method of any of Embodiments 33-58, wherein the first antibody variant is a human monoclonal antibody or a humanized monoclonal antibody variant.

Embodiment 60. The method of any of Embodiments 33-59, wherein the first antibody variant is an IgG antibody variant.

Embodiment 61. The method of Embodiment 60, wherein the IgG antibody variant is selected from the group consisting of an IgG1, IgG2, IgG3, and IgG4 antibody variant.

Embodiment 62. The method of Embodiment 61, wherein the IgG antibody variant is an IgG1 antibody variant.

Embodiment 63. The method of Embodiment 61, wherein the IgG antibody variant is an IgG2 antibody variant.

Embodiment 64. The method of Embodiment 61, wherein the IgG antibody variant is an IgG3 antibody variant.

Embodiment 65. The method of Embodiment 61, wherein the IgG antibody variant is an IgG4 antibody variant.

Embodiment 66. A method of increasing stability of a first antibody or first antibody variant, comprising
a. identifying the germline original amino acid sequence for the heavy chain of the first antibody or of the antibody portion of the antibody variant;
b. identifying the amino acid residues at heavy chain position 56 (AHo numbering) and heavy chain position 80 (AHo numbering) in the first antibody or of the antibody portion of the antibody variant; and
c. substituting at heavy chain positions 56 and 80 the identified residues from the germline original amino acid sequence in the first antibody or of the antibody portion of the antibody variant, thereby creating a second antibody or second antibody variant,
wherein the second antibody is more stable than the unsubstituted first antibody, or wherein the second antibody variant is more stable than the unsubstituted first antibody variant.

Embodiment 67. The method of Embodiment 66, wherein the increased stability of the second antibody or second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

Embodiment 68. The method of Embodiment 67, wherein the increase in titer is measured by the rate of binding to a protein A coated probe tip using an Octet Forte Bio Instrument.

Embodiment 69. The method of Embodiment 67, wherein the increased yield is measured by protein A or protein G capture.

Embodiment 70. The method of Embodiment 67, wherein the increased purity is measured by SEC of purified protein.

Embodiment 71. The method of Embodiment 67, wherein the reduction in high molecular weight species is measured by SEC and area under the curve for peaks at each molecular weight Embodiment 72. The method of Embodiment 67, wherein the increased melting point temperature is measured by DSF or DSC.

Embodiment 73. The method of Embodiment 67, wherein the increased temperature of aggregation is measured by DSF.

Embodiment 74. The method of Embodiment 67, wherein the increased temperature of the onset of melting is measured by DSF.

Embodiment 75. The method of any one of Embodiments 66-74, wherein the first antibody is a monoclonal antibody.

Embodiment 76. The method of Embodiment 75, wherein the first antibody is a human monoclonal antibody or a humanized monoclonal antibody.

Embodiment 77. The method of any one of Embodiments 66-76, wherein the first antibody is an IgG antibody.

Embodiment 78. The method of Embodiment 77, wherein the IgG antibody is selected from the group consisting of an IgG1, IgG2, IgG3, and IgG4 antibody.

Embodiment 79. The method of Embodiment 78, wherein the IgG antibody is an IgG1 antibody.

Embodiment 80. The method of Embodiment 78, wherein the IgG antibody is an IgG2 antibody.

Embodiment 81. The method of Embodiment 78, wherein the IgG antibody is an IgG3 antibody.

Embodiment 82. The method of Embodiment 78, wherein the IgG antibody is an IgG4 antibody.

Embodiment 83. The method of any of Embodiments 66-82, wherein the first antibody variant is a multi-specific antibody.

Embodiment 84. The method of Embodiment 83, wherein the multi-specific antibody is a bispecific antibody or trispecific antibody.

Embodiment 85. The method of any of Embodiments 66-84, wherein the first antibody variant is an antibody fragment that can bind an antigen.

Embodiment 86. The method of Embodiment 85, wherein the antibody fragment is selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

Embodiment 87. The method of any of Embodiments 66-86, wherein the first antibody variant is a human monoclonal antibody or a humanized monoclonal antibody variant.

Embodiment 88. The method of any of Embodiments 66-87, wherein the first antibody variant is an IgG antibody variant.

Embodiment 89. The method of any of Embodiments 66-88, wherein the IgG antibody variant is selected from the group consisting of an IgG1, IgG2, IgG3, and IgG4 antibody variant.

Embodiment 90. The method of Embodiment 89, wherein the IgG antibody variant is an IgG1 antibody variant.

Embodiment 91. The method of Embodiment 89, wherein the IgG antibody variant is an IgG2 antibody variant.

Embodiment 92. The method of Embodiment 89, wherein the IgG antibody variant is an IgG3 antibody variant.

Embodiment 93. The method of Embodiment 89, wherein the IgG antibody variant is an IgG4 antibody variant.

Embodiment 94. The method of any of Embodiments 1-93, wherein the second antibody or second antibody variant is substituted with any one of the following pairs of residues at positions 56 and 80 of the heavy chain (AHo numbering), respectively: GF, GI, GL, GT, GV, AF, AI, AL, AV, AA, AM, SA, SI, or ST. By way of example, under this nomenclature “GF” would refer to a “G” at position 56 and an “F” at position 80 (AHo numbering).

Embodiment 95. The method of any of Embodiments 1-94, further comprising formulating the second antibody or second antibody variant into a pharmaceutical composition.

Embodiment 96. An antibody or antibody variant made by the method of any of Embodiments 1-95.

Embodiment 97. A pharmaceutical composition comprising the antibody or antibody variant of Embodiment 96.

The following Examples section is given solely by way of example and are not set forth to limit the disclosure or claims in any way.

### EXAMPLES

### Example 1—Experimental Design

Antibody mutations were implemented by designing codon changes and having nucleotides synthesized accordingly. Modified fragments were integrated into the open reading frame using Golden Gate cloning methods (Engler et al 2009, Engler et al 2008). Antibodies were expressed in HEK 293-6E cells (a suspension cell line that expresses a truncated variant of the Epstein Barr virus nuclear antigen (EBNA) 1 (Durocher et al 2002), unless otherwise noted. The produced antibodies were purified using protein A attached to solid supports.

The following germline families were examined: VH1/VK4, VH4/VL1, VH3/VK3, VH6/VK1, and VH2/VL2. In the heavy chain, 56A and 56G were tested, as was 80I and 80M.

The measured parameters were:
Titer at harvest (mg/L)
Melting temperature (Tm; as measured by differential scanning fluorimetry (DSF) or differential scanning calorimetry (DSC), the latter giving two outputs: Tm1 and Tm2)
Yield (as measured by antibody captured by protein A)
High molecular weight (HMW) species as determined by size-exclusion chromatography (SEC) and peak molecular weight (Mp, defined as the molecular weight of the highest peak).

### Example 2—VH1 Germline

In this example, mutations in an antibody, mAb1 (an IgG1) that originated in VH1 germline (VH1/VK4) were examined, and the antibodies assayed as described in Example 1.

TABLE 2.1
Observations for a monoclonal antibody from VH1/VK4 germline, mAb1

| | | | Titer | Lot Yield | Yield | MP Purity | HMW | | | |
|---|---|---|---|---|---|---|---|---|---|---|
| Ab | 56⁺ | 80⁺ | (mg/L) | (mg/L) | (%) | (%) | (%) | Tm | Tagg | Tonset |
| mAb1GM\hat{ } | G* | M* | 117.3 | 75 | 63.9 | ND | ND | 75.1** | 73.3 | 66.7 |
| mAb1GI | G | I | 72.8 | 35 | 48.0 | ND | ND | 72.0 | 73.1 | 64.1 |
| mAb1AM | A | M | 9.48 | 4.0 | 42.2 | 97.4 | 2.6 | ND | ND | ND |
| mAb1AI | A | I | 12.3 | 4.6 | 37.4 | 97.8 | 2.2 | ND | ND | ND |
| mAb1^{~} | G | L | ND | ND | ND | ND | ND | 76.5, 83.6^{&} | | |
| mAb1 | G | L | ND | ND | ND | ND | ND | 74.8 | 72.6 | 62.3 |

⁺denotes amino acid at the indicated position (AHo numbering)
*indicates germline residue at the indicated position
\hat{ }mAb1 = “wild-type” of mAb1. The suffixes indicate the residues found at positions 56 and 80, respectively.
ND, no data
^{~}a variant of mAb1 that has the wild-type residues (G and L) at positions 56 and 80, respectively; data were derived from a separate set of experiments
^{&}Data were derived from DSC experiments and represent Tm1 and Tm2.
**When measured by DSC, Tm1 was 76.9, and Tm2 was 84.4

From these experiments, when mAb1 had A at position 56 and M at position 80, the antibody (mAb1AM) had the lowest titer observed of the tested antibodies. Similarly, mAb1AI also had low titer. However, mAb1GM had the highest titer observed of the tested antibodies; mAb1GI had the second highest titer.

### Example 3—VH3 Germline

In this example, mutations in an antibody, mAb2 (an IgG2 Ab), that originated from the VH3 germline (VH3/VK3) were examined, and the antibodies assayed as described in Example 1.

TABLE 3.1
Observations for a monoclonal antibody from VH3/VK3 germline, mAb2

| | | | Titer | Lot Yield | Yield | MP Purity | HMW | | | |
|---|---|---|---|---|---|---|---|---|---|---|
| Ab | 56⁺ | 80⁺ | (mg/L) | (mg/L) | (%) | (%) | (%) | Tm | Tagg | Tonset |
| mAb2GI | G | I | 533.4 | 254 | 47.6 | 98.9 | 1.1 | 75.6 | ND | ND |
| mAb2GM | G | M | 525.9 | 262 | 49.9 | 99.3 | 0.7 | 73.2 | ND | ND |
| mAb2AM | A | M | 329.4 | 143 | 43.5 | 99.1 | 0.9 | 70.1 | ND | ND |
| mAb2 | A* | I* | 533.2 | 249 | 46.7 | 98.3 | 1.7 | 76.0 | ND | ND |

⁺denotes amino acid at the indicated position (AHo numbering)
*indicates germline residue at the indicated position
ND, no data

From these experiments, when mAb2 had A at position 56 and M at position 80, the antibody (mAb2AM) had the lowest titer observed of the tested antibodies, as well as the lowest melting point (Tm), suggesting that this antibody was less stable. However, mAb2AI and mAb2GM had higher titers and melting points of the tested antibodies.

### Example 4—VH4 Germline

In this example, mutations in an antibody, mAb3 (an IgG1 Ab), that originated from the VH4 germline (VH4/VL1) were examined, and the antibodies assayed as described in Example 1.

TABLE 4.1
Observations for a monoclonal antibody from VH4/VL1 germline, mAb3

| | | | Titer | Lot Yield | Yield | MP Purity | HMW | | | |
|---|---|---|---|---|---|---|---|---|---|---|
| Ab | 56⁺ | 80⁺ | (mg/L) | (mg/L) | (%) | (%) | (%) | Tm | Tagg | Tonset |
| mAb3 | G* | I* | 244.1 | 90 | 36.9 | | | 70.9 | 73.3 | 65.3 |
| mAb3AI | A | I | 105.3 | 46 | 43.8 | | | 65.4 | 72.4 | 57.9 |
| mAb3GM | G | M | 292 | 134 | 45.9 | | | 70.4 | 79.1 | 64.9 |
| mAb3AM | A | M | 177.7 | 79 | 44.6 | | | 65.4 | 74.2 | 57.7 |

⁺denotes amino acid at the indicated position (AHo numbering)
*indicates germline residue at the indicated position

From these experiments, when mAb3 had A at position 56 and M or I at position 80, the antibodies (mAb3AM and mAb3AI) had the lowest titers observed of the tested antibodies, as well as the lowest melting points (Tm), suggesting that these antibodies were less stable. However, mAb3(GI) and mAb3GM had higher titers and melting points of the tested antibodies.

### Example 5—VH6 Germline

In this example, mutations in an antibody, mAb4 (an IgG1 Ab), that originated from the VH6 germline (VH6/VK1) were examined, and the antibodies assayed as described in Example 1.

TABLE 5.1
Observations for a monoclonal antibody from VH6/VK1 germline, mAb4

| | | | Titer | Lot Yield | Yield | MP Purity | HMW | | | |
|---|---|---|---|---|---|---|---|---|---|---|
| Ab | 56⁺ | 80⁺ | (mg/L) | (mg/L) | (%) | (%) | (%) | Tm | Tagg | Tonset |

| | | | | | | | | |
|---|---|---|---|---|---|---|---|---|
| mAb4AI | A | I | 20.5 | 2.7 | 13.2 | 96.3 | 1.4 | 71.1 |
| mAb4AM | A | M | 3.53 | 0.3 | 8.5 | 95.5 | 2.4 | ND |
| mAb4GM | G | M | 66.8 | 11.6 | 17.3 | 96.6 | 1.0 | 70.5 |
| mAb4 | G* | I* | 185.5 | 71 | 38.3 | 99.0 | 1.0 | 73.7 |

⁺denotes amino acid at the indicated position (AHo numbering)
*indicates germline residue at the indicated position
ND, no data

From these experiments, when mAb4 had A at position 56 and M at position 80, the antibody (mAb4AM) had the lowest titer observed of the tested antibodies; the melting point was not determined. However, mAb4(GI) (germline) and mAb4GM had higher titers and melting points of the tested antibodies, with mAb4GI being superior to mAb4GM.

### Example 6—VH2 Germline

In this example, mutations in an antibody, mAb5 (an IgG1 Ab), that originated from the VH2 germline (VH2/VL2) were examined, and the antibodies assayed as described in Example 1.

TABLE 6.1
Observations for a monoclonal antibody from VH2/VL2 germline, mAb5

| | | | Titer | Lot Yield | Yield | MP Purity | HMW | | | |
|---|---|---|---|---|---|---|---|---|---|---|
| Ab | 56⁺ | 80⁺ | (mg/L) | (mg/L) | (%) | (%) | (%) | Tm | Tagg | Tonset |
| mAb5 | A* | I* | 220 | 174 | 79.0 | 98.0 | 2.0 | 78.2 | | |
| mAb5GM | G | M | 220.8 | 94 | 42.7 | 98.2 | 1.8 | 73.0 | | |

⁺denotes amino acid at the indicated position (AHo numbering)
*indicates germline residue at the indicated position

From these experiments, both tested antibodies had high titers and favorable melting points.

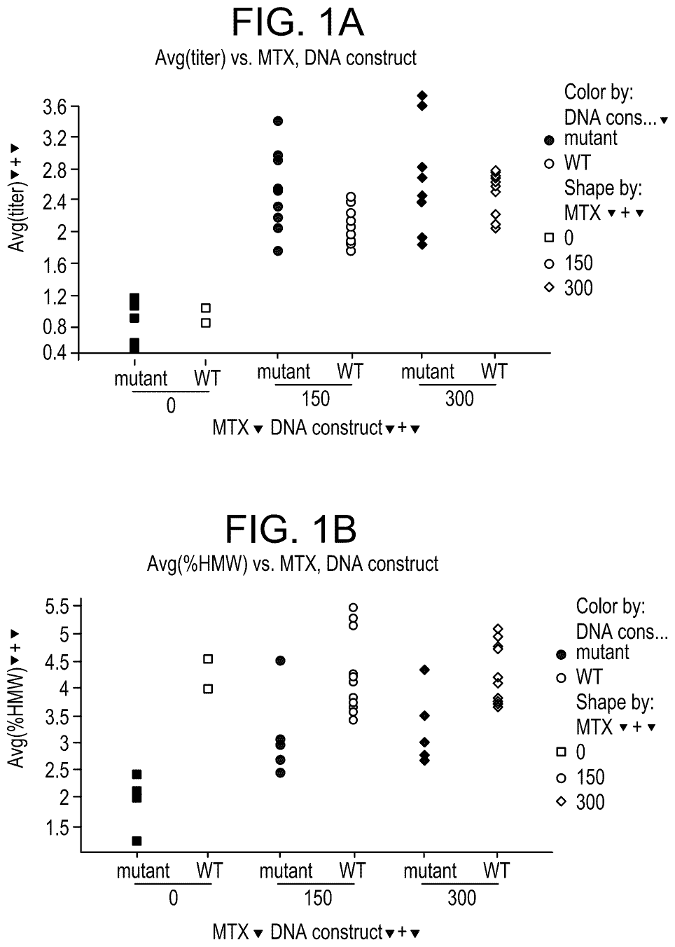

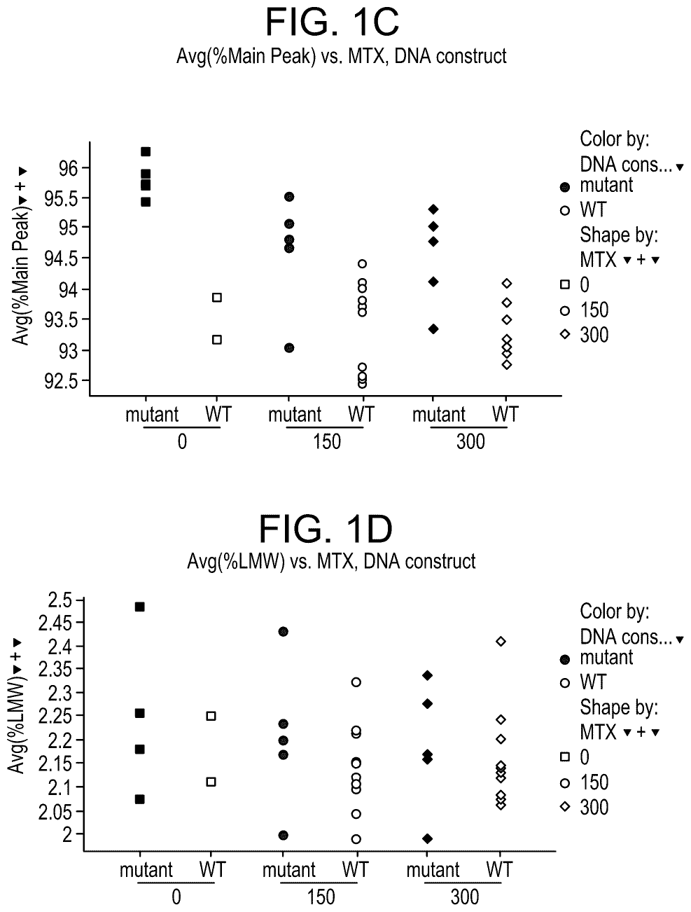

When these antibodies were expressed by Chinese Hamster Ovary (CHO) cells, mAb5GM (“mutant” in FIG. 1) had higher titer (FIG. 1A), less HMW species (FIG. 1B) and more MP purity (FIG. 1C) across clones and MTX levels compared to mAb5(AI) (germline; “WT” in FIG. 1) antibody.

### Example 7—Comparison of Antibody Titer by IgG Sub-Type and HC56 and HC80 Mutations

The harvest titers of mAb1 and mAb3 (the latter in different sub-types) were determined. Table 7.1 shows the results. In this experiment, the heavy chain variant HC:56G, HC:80M showed the most desirable final growth characteristics. The HC:56G, HC:80M variant also produced higher titers (day 6) in IgG1, IgG2 and IgG4 molecules, with the exception of mAb3 (IgG4 format); instead, this molecule displayed the highest titers from the HC:56A, HV:80M set of substitutions.

TABLE 7.1
Harvest titer of IgG sub-types with
various HC56 and HC80 substitutions

| | HC amino acid | HC amino acid | Harvest Titer |
|---|---|---|---|
| Description | position 56 | position 80 | (mg/L) |
| mAb1 (IgG1) | G | L | 61.7 |
| mAb1 (IgG1) | G | M | 14.1 |
| mAb1 (IgG1) | G | I | 3.96 |
| mAb1 (IgG1) | A | M | 0 |
| mAb1 (IgG1) | A | I | 5.09 |
| mAb3 (IgG1) | G | I | 42.8 |
| mAb3 (as IgG2) | G | I | 20.3 |
| mAb3 (as IgG4) | G | I | 19.8 |
| mAb3 (IgG1) | A | I | 68.9 |
| mAb3 (as IgG2) | A | I | 31 |
| mAb3 (as IgG4) | A | I | 38.4 |
| mAb3 (IgG1) | G | M | 241.8 |
| mAb3 (as IgG2) | G | M | 147.7 |
| mAb3 (as IgG4) | G | M | 45.2 |
| mAb3 (IgG1) | A | M | 12.4 |
| mAb3 (as IgG2) | A | M | 6.49 |
| mAb3 (as IgG4) | A | M | 79.2 |

### Example 8—Identification of Amino Acids for IgG1 HC:56 and HC:80

In this Example, additional options for amino acids that can be used at IgG1 HC:56 and HC:80 were identified using two methods, one being an in-depth phylogenetic analysis and screen, and the other being based on Rosetta modeling of all 400 H:56 H:80 combinations. It is noted that these experiments provide additional testing and analysis of the possible options at HC: 56 and HC:80. Amino acid pairings with moderate results in one or more computational screen were mutated into the model molecules before expression levels upon harvest from 293-6E cell cultures and Tm by DSF were compared.

For the phylogenetic analysis, a blast search of mAb 1 and mAb 2 was conducted in Protein NR (e=0.0001). The ˜10,000 sequences that were identified for each molecule were CD-hit clustered and the most common residues were identified and ranked according to frequency. Top amino acids were cloned into mAb 1 and mAb 2.

For the phylogenetic analysis of mAb 1, the most common residue pairs are shown in Table 8.1 below (bolded residue pairs were tested; asterisks denote germline residues):

TABLE 8.1

| H56 | H80 |
|---|---|
| A | I |
| A | L |
| A | M |
| A | V |
| G | F |
| G | I |
| G | L |
| G* | M* |
| G | V |
| S | I |
| S | V |

For the phylogenetic analysis of mAb 2, the most common residue pairs are shown in Table 8.2 below (bolded residue pairs were tested):

TABLE 8.2

| H56 | H80 |
|---|---|
| A | I |
| A | V |
| G | F |
| G | I |
| G | L |
| G | M |
| G | V |
| S | I |
| M | V |
| T | I |
| V | I |

For the Rosetta analysis, Rosetta software was used to measure energy scores of all amino acid combinations at HC:56 and HC:80 in mAb 1 and mAb 2. Without being limited by theory, it is contemplated that under this approach, Rosetta attempted all of the 400 possible H56:H80 amino acid combinations using the standard genetically-encoded amino acids. From the Rosetta analysis, HC:56 and HC:80 amino acid combinations were compared according to the Rosetta “total score” and “p_aa_pp” score and ranked by a 1:1 weighted z-score. Variants with favorable energy scores that were not already in the phylogenic analysis were cloned into mAb 1 and mAb 2 for further testing.

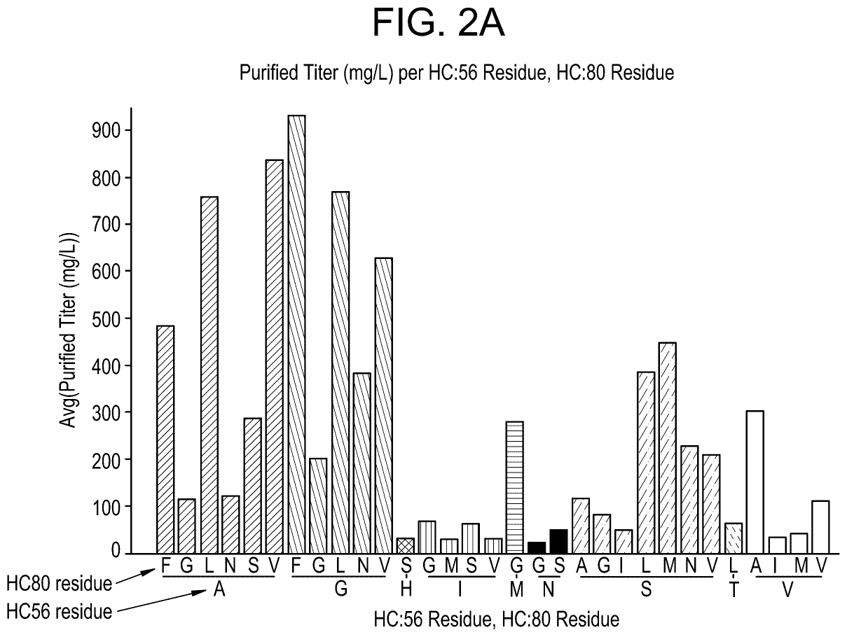

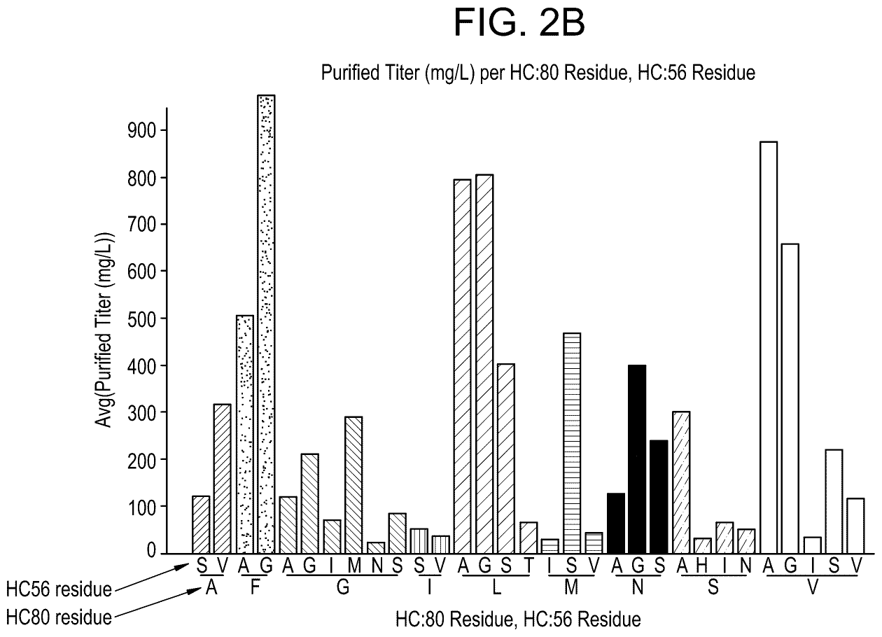

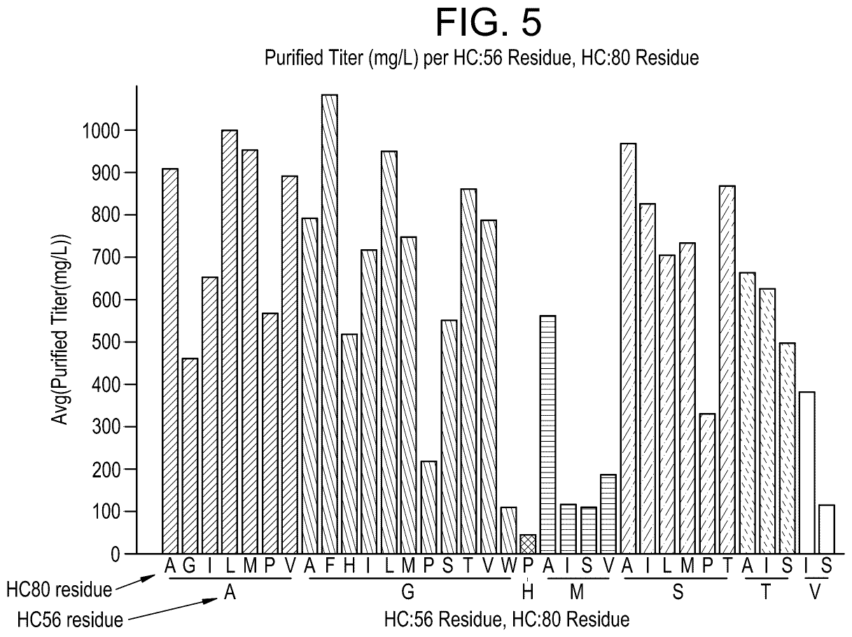

At HC:56, results for mAb 1 indicate that the highest titers could be achieved with HC:56G or HC:56A, followed by HC:56S (See, e.g., FIGS. 2A-2B). Results for mAb 2 indicate that the highest titers could be achieved with HC:56A, HC:56G, or HC:56S (See, e.g., FIG. 5). Without being limited by theory, it is noted that these residues are most similar in their relatively small size when compared with the full range of amino acids.

At HC:80, results for mAb 1 indicate that the highest titers could be achieved with HC:80F, HC:80L, or HC:80V. Results for mAb 2 indicate that the highest titers could be achieved with HC:80L, HC:80M, HC:80A, HC:80V, HC:80F, HC:80I. Also, in two cases, HC:80T resulted in relatively high expression. Without being limited by theory, a generalization of these results is that the use of hydrophobic residues at HC:80 results in higher titers.

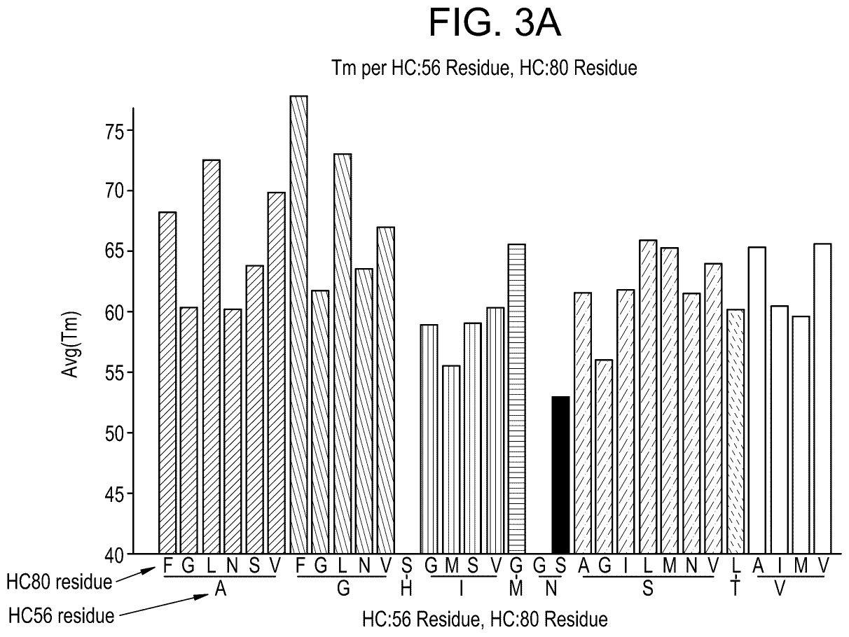

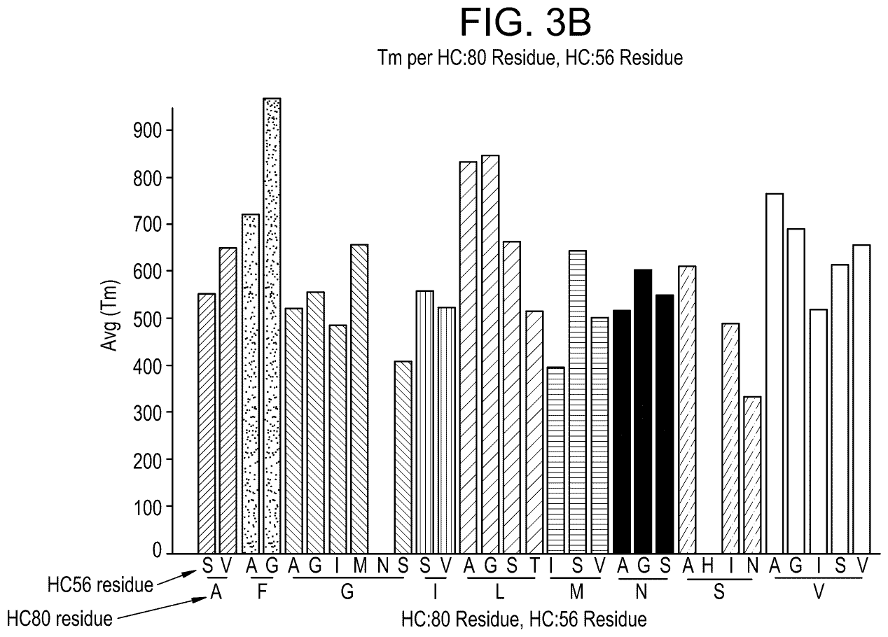

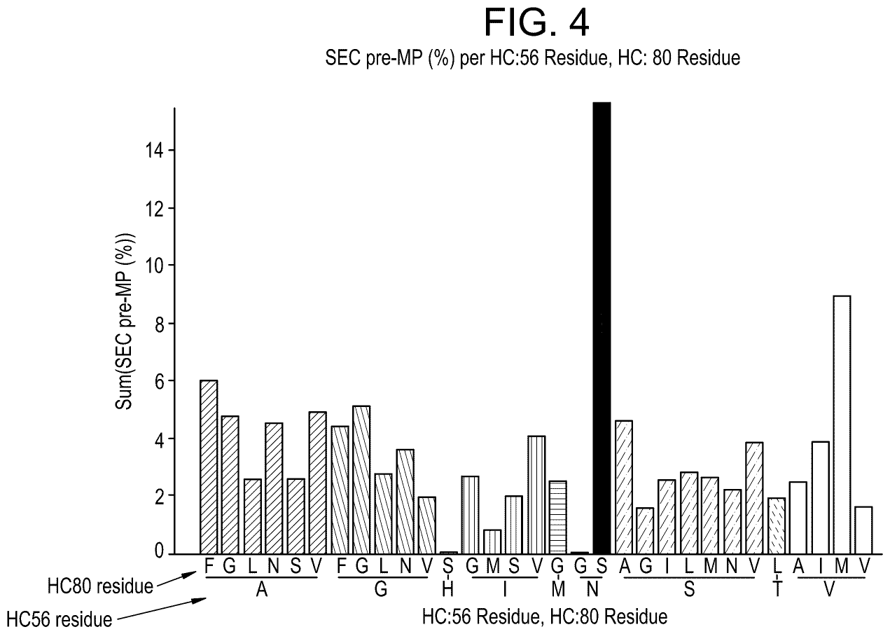

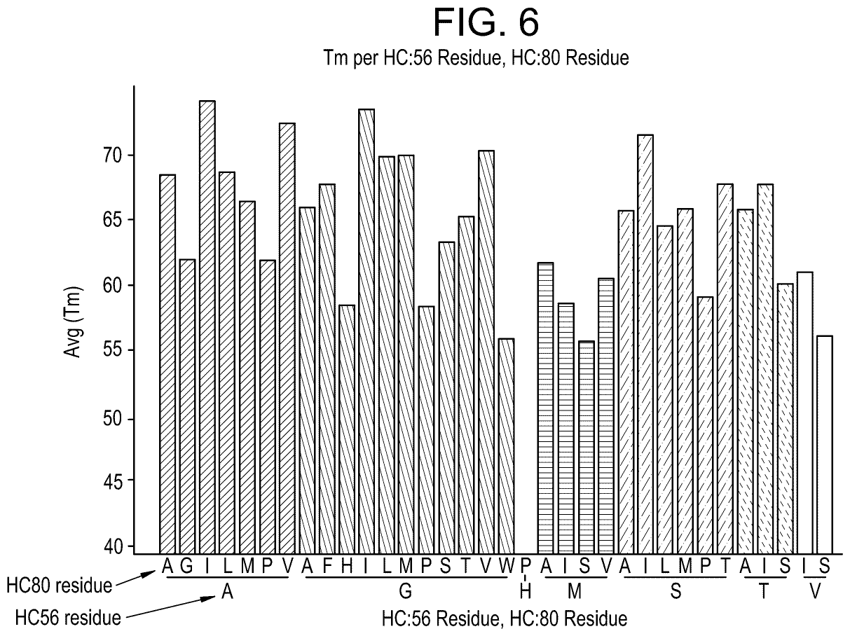

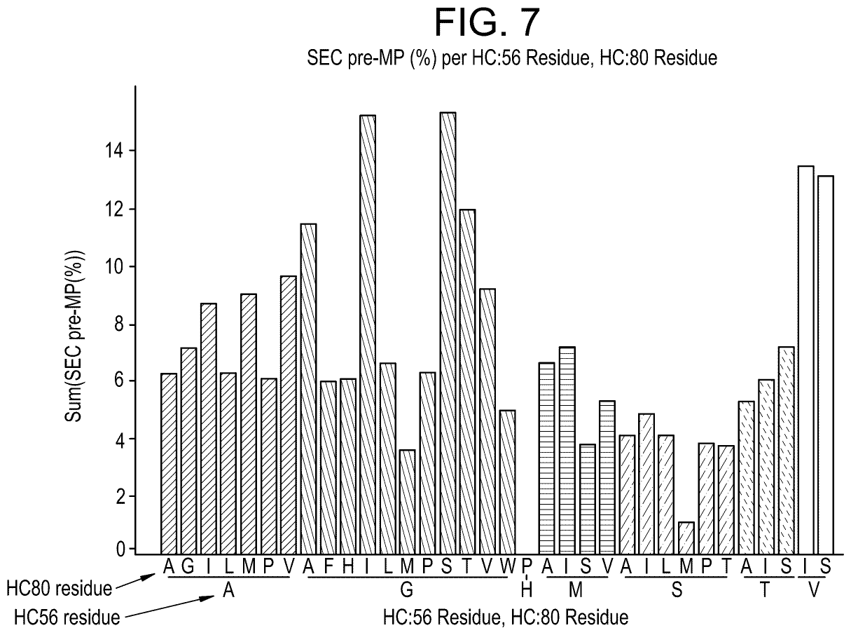

Among the engineered variants of mAb 1, the six variants with the highest titer correlated with the six variants with the highest Tm (See FIGS. 2A-2B and 3A-3B). Among variants of mAb 2, the differences in Tm were less pronounced, though the low titer variants continued to have low Tm's (See FIG. 6). In addition, the use of hydrophobic residues at HC:80 correlated with higher Tm's.

Effects of combinations of residues at heavy chain position 56 and heavy chain position 80 (AHo numbering) in each of mAb 1 and mAb 2 are summarized in Table 8.3 below. In the nomenclature of Table 8, the first residue refers to heavy chain position 56 (AHo numbering), and the second residue refers to heavy chain position 80 (AHo numbering). Thus, by way of example “GF” would refer to a “G” at heavy chain position 56 (AHo numbering) and an “F” at heavy chain position 80 (AHo numbering):

TABLE 8.3

| mAb 1 | Top Titers | GF, GL, GV, AF, AL, AV |
|---|---|---|
| | Top Tm | GF, GL, GV, AF, AL, AV |
| | (Low titers were observed with HC: 56 H, I, N, and T); | |
| | (Aggregation at low temperature was observed | |
| | with SN, SA, and TL) | |
| mAb 2 | Top Titers | AA, AL, AM, AV, GF, GL, GT, SA, ST |
| | Top Tm | AI, AV, GI, SI, GV |

(low titers we observed with HC: 56 H and M)

### REFERENCES

Bentley D R, Balasubramanian S, Swerdlow H P, Smith G P, Milton J, et al. 2008. Accurate whole human genome sequencing using reversible terminator chemistry. Nature 456: 53-9
Branton D, Deamer D W, Marziali A, Bayley H, Benner S A, et al. 2008. The potential and challenges of nanopore sequencing. Nat Biotechnol 26: 1146-53
Durocher Y, Perret S, Kamen A. 2002. High-level and high-throughput recombinant protein production by transient transfection of suspension-growing human 293-EBNA1 cells. Nucleic Acids Res 30: E9
Eid J, Fehr A, Gray J, Luong K, Lyle J, et al. 2009. Real-time DNA sequencing from single polymerase molecules. Science 323: 133-8
Engler C, Gruetzner R, Kandzia R, Marillonnet S. 2009. Golden gate shuffling: a one-pot DNA shuffling method based on type IIs restriction enzymes. PLoS One 4: e5553
Engler C, Kandzia R, Marillonnet S. 2008. A one pot, one step, precision cloning method with high throughput capability. PLoS One 3: e3647
Fahrner R L, Knudsen H L, Basey C D, Galan W, Feuerhelm D, et al. 2001. Industrial purification of pharmaceutical antibodies: development, operation, and validation of chromatography processes. Biotechnol Genet Eng Rev 18: 301-27
Honegger A, Pluckthun A. 2001. Yet another numbering scheme for immunoglobulin variable domains: an automatic modeling and analysis tool. J Mol Biol 309: 657-70
Jafari R, Almqvist H, Axelsson H, Ignatushchenko M, Lundback T, et al. 2014. The cellular thermal shift assay for evaluating drug target interactions in cells. Nat Protoc 9: 2100-22
Kamerzell T J, Esfandiary R, Joshi S B, Middaugh C R, Volkin D B. 2011. Protein-excipient interactions: mechanisms and biophysical characterization applied to protein formulation development. Adv Drug Deliv Rev 63: 1118-59
Kelley B. 2009. Industrialization of mAb production technology: the bioprocessing industry at a crossroads. MAbs 1: 443-52
Kostelny S A, Cole M S, Tso J Y. 1992. Formation of a bispecific antibody by the use of leucine zippers. J Immunol 148: 1547-53
Lo M C, Aulabaugh A, Jin G, Cowling R, Bard J, et al. 2004. Evaluation of fluorescence-based thermal shift assays for hit identification in drug discovery. Anal Biochem 332: 153-9
Makhatadze G. 1998. Measuring protein thermostability by differential scanning calorimetry. Curr. Protocols Protein Sci. 12: 7.9.1-7.9.14
Margulies M, Egholm M, Altman W E, Attiya S, Bader J S, et al. 2005. Genome sequencing in microfabricated high-density picolitre reactors. Nature 437: 376-80
Mason M, Sweeney B, Cain K, Stephens P, Sharfstein S T. 2012. Identifying bottlenecks in transient and stable production of recombinant monoclonal-antibody sequence variants in Chinese hamster ovary cells. Biotechnol Prog 28: 846-55
Medzihradszky K F, Chalkley R J. 2015. Lessons in de novo peptide sequencing by tandem mass spectrometry. Mass Spectrom Rev 34: 43-63
Minde D P, Maurice M M, Rudiger S G. 2012. Determining biophysical protein stability in lysates by a fast proteolysis assay, FASTpp. PLoS One 7: e46147
Nyren P, Lundin A. 1985. Enzymatic method for continuous monitoring of inorganic pyrophosphate synthesis. Anal Biochem 151: 504-9
Pantoliano M W, Petrella E C, Kwasnoski J D, Lobanov V S, Myslik J, et al. 2001. High-density miniaturized thermal shift assays as a general strategy for drug discovery. J Biomol Screen 6: 429-40
Powell M F, Nguyen T, Baloian L. 1998. Compendium of excipients for parenteral formulations. PDA journal of pharmaceutical science and technology 52: 238-311
Quax T E, Claassens N J, Soll D, van der Oost J. 2015. Codon Bias as a Means to Fine-Tune Gene Expression. Mol Cell 59: 149-61
Ronaghi M, Uhlen M, Nyren P. 1998. A sequencing method based on real-time pyrophosphate. Science 281: 363, 65
Rothberg J M, Hinz W, Rearick T M, Schultz J, Mileski W, et al. 2011. An integrated semiconductor device enabling non-optical genome sequencing. Nature 475: 348-52
Sanger F, Nicklen S, Coulson A R. 1977. DNA sequencing with chain-terminating inhibitors. Proc Natl Acad Sci USA 74: 5463-7
Semisotnov G V, Rodionova N A, Razgulyaev O I, Uversky V N, Gripas A F, Gilmanshin R I. 1991. Study of the “molten globule” intermediate state in protein folding by a hydrophobic fluorescent probe. Biopolymers 31: 119-28
Songsivilai S, Lachmann P J. 1990. Bispecific antibody: a tool for diagnosis and treatment of disease. Clin Exp Immunol 79: 315-21
Valouev A, Ichikawa J, Tonthat T, Stuart J, Ranade S, et al. 2008. A high-resolution, nucleosome position map of C. elegans reveals a lack of universal sequence-dictated positioning. Genome Res 18: 1051-63
Wu X, Demarest S J. 2018. Building blocks for bispecific and trispecific antibodies. Methods
Wu X, Yuan R, Bacica M, Demarest S J. 2018. Generation of orthogonal Fab-based trispecific antibody formats. Protein Eng Des Sel 31: 249-56

Both the foregoing general description and the following detailed description are exemplary and explanatory only and are not restrictive. The use of the singular includes the plural unless specifically stated otherwise. The use of “or” means “and/or” unless stated otherwise. The use of the term “including”, as well as other forms, such as “includes” and “included”, is not limiting. Terms such as “element” or “component” encompass both elements and components comprising one unit and elements and components that comprise more than one subunit unless specifically stated otherwise. The use of the term “portion” can include part of a moiety or the entire moiety. When a numerical range is mentioned, e.g., 1-5, all intervening values are explicitly included, such as 1, 2, 3, 4, and 5, as well as fractions thereof, such as 1.5, 2.2, 3.4, and 4.1.

“About” or “˜” means, when modifying a quantity (e.g., “about” 3 mM), that variation around the modified quantity can occur. These variations can occur by a variety of means, such as typical measuring and handling procedures, inadvertent errors, ingredient purity, and the like.

“Comprising” and “comprises” are intended to mean that the formulations and methods include the listed elements but do not exclude other unlisted elements. The terms “consisting essentially of” and “consists essentially of,” when used in the disclosed methods include the listed elements, exclude unlisted elements that alter the basic nature of the formulation and/or method, but do not exclude other unlisted elements. A formulation consisting essentially of elements would not exclude trace amounts of other elements, such as contaminants from any isolation and purification methods or pharmaceutically acceptable carriers (e.g., phosphate buffered saline), preservatives, and the like, but would exclude, for example, additional unspecified amino acids. The terms “consisting of” and “consists of” when used to define formulations and methods exclude more than trace elements of other ingredients and substantial method steps for administering the compositions described herein. Embodiments defined by each of these transition terms are within the scope of this disclosure.

1. A method of increasing stability of a first antibody, comprising substituting glycine, alanine, or serine at heavy chain position 56 (AHo numbering) to create a second antibody, wherein the second antibody is more stable than the unsubstituted first antibody.

2. The method of claim 1, wherein the glycine is substituted at heavy chain position 56.

3. The method of any one of claims 1-2, wherein the second antibody is further substituted with a hydrophobic amino acid residue at heavy chain position 80 (AHo numbering).

4. The method of claim 3, wherein the hydrophobic amino acid residue is selected from the group consisting of: alanine, isoleucine, phenylalanine, leucine, methionine, and valine.

5. The method of claim 3, wherein the hydrophobic amino acid residue is selected from the group consisting of: phenylalanine, leucine, and valine.

6. The method of any one of claims 1-2, wherein the second antibody is further substituted with methionine at position 80 (AHo numbering).

7. The method of any one of claims 1-2, wherein the second antibody is further substituted with isoleucine at position 80 (AHo numbering).

8. A method of increasing stability of a first antibody, comprising substituting a hydrophobic amino acid residue at heavy chain position 80 (AHo numbering) of the first antibody to create a second antibody, wherein the second antibody is more stable than the unsubstituted first antibody.

9. The method of claim 8, wherein the hydrophobic amino acid residue is selected from the group consisting of: alanine, isoleucine, phenylalanine, leucine, methionine, and valine.

10. The method of claim 8, wherein the hydrophobic amino acid residue is selected from the group consisting of: phenylalanine, leucine, and valine.

11. A method of increasing stability of a first antibody, comprising substituting alanine, phenylalanine, isoleucine, leucine, methionine, threonine, or valine at heavy chain position 80 (AHo numbering) of the first antibody to create a second antibody, wherein the second antibody is more stable than the unsubstituted first antibody.

12. The method of claim 11, wherein the methionine is substituted at heavy chain position 80 of the first antibody.

13. The method of claim 11, wherein the isoleucine is substituted at heavy chain position 80 of the first antibody.

14. The method of any one of claims 8-13, wherein the second antibody is further substituted with alanine, glycine, or serine at heavy chain position 56 (AHo numbering).

15. The method of any one of claims 8-13, wherein the second antibody is further substituted with alanine or glycine at heavy chain position 56 (AHo numbering).

16. The method of any one of claims 8-13, wherein the second antibody is further substituted with glycine at heavy chain position 56 (AHo numbering)

17. The method of any of claims 1-16, wherein the increased stability of the second antibody is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

18. The method of claim 17, wherein the increase in titer is measured by the rate of binding to a protein A coated probe tip using an Octet Forte Bio Instrument.

19. The method of claim 17, wherein the increased yield is measured by protein A or protein G capture.

20. The method of claim 17, wherein the increased purity is measured by size-exclusion chromatography (SEC) of the purified antibodies.

21. The method of claim 17, wherein the reduction in high molecular weight species is measured by size-exclusion chromatography (SEC) and area under the curve for each peak at each molecular weight.

22. The method of claim 17, wherein the increased melting point temperature is measured by differential scanning fluorimetry (DSF) or differential scanning calorimetry (DSC).

23. The method of claim 17, wherein the increased temperature of aggregation is measured by DSF.

24. The method of claim 17, wherein the increased temperature of the onset of melting is measured by DSF.

25. The method of any preceding claim, wherein the first antibody is a monoclonal antibody.

26. The method of any preceding claim, wherein the first antibody is a human monoclonal antibody or a humanized monoclonal antibody.

27. The method of any preceding claim, wherein the first antibody is an IgG antibody.

28. The method of claim 27, wherein the IgG antibody is selected from the group consisting of an IgG1, IgG2, IgG3, and IgG4 antibody.

29. The method of claim 27, wherein the IgG antibody is an IgG1 antibody.

30. The method of claim 27, wherein the IgG antibody is an IgG2 antibody.

31. The method of claim 27, wherein the IgG antibody is an IgG3 antibody.

32. The method of claim 27, wherein the IgG antibody is an IgG4 antibody.

33. A method of increasing stability of a first antibody variant, comprising substituting glycine, alanine, or serine at heavy chain position 56 (AHo numbering) to create a second antibody variant, wherein the second antibody variant is more stable than the unsubstituted first antibody variant.

34. The method of claim 33, wherein the glycine is substituted at heavy chain position 56.

35. The method of any one of claims 33-34, wherein the second antibody variant is further substituted with a hydrophobic amino acid residue at heavy chain position 80 (AHo numbering).

36. The method of claim 35, wherein the hydrophobic amino acid residue is selected from the group consisting of: alanine, isoleucine, phenylalanine, leucine, methionine, and valine.

37. The method of claim 35, wherein the hydrophobic amino acid residue is selected from the group consisting of: phenylalanine, leucine, and valine.

38. A method of increasing stability of a first antibody variant, comprising substituting a hydrophobic amino acid residue at heavy chain position 80 (AHo numbering) of the first antibody variant to create a second antibody variant, wherein the second antibody variant is more stable than the unsubstituted first antibody variant.

39. The method of claim 38, wherein the hydrophobic amino acid residue is selected from the group consisting of: alanine, isoleucine, phenylalanine, leucine, methionine, and valine.

40. The method of claim 38, wherein the hydrophobic amino acid residue is selected from the group consisting of: phenylalanine, leucine, and valine.

41. A method of increasing stability of a first antibody variant, comprising substituting alanine, phenylalanine, isoleucine, leucine, methionine, threonine, or valine at heavy chain position 80 (AHo numbering) of the first antibody variant to create a second antibody variant, wherein the second antibody variant is more stable than the unsubstituted first antibody variant.

42. The method of claim 41, wherein the methionine is substituted at heavy chain position 80 of the first antibody variant.

43. The method of claim 41, wherein the isoleucine is substituted at heavy chain position 80 of the first antibody variant.

44. The method of any one of claims 38-43, wherein the second antibody variant is further substituted with alanine, glycine, or serine at heavy chain position 56 (AHo numbering).

45. The method of any one of claims 38-43, wherein the second antibody variant is further substituted with alanine or glycine at heavy chain position 56 (AHo numbering).

46. The method of any one of claims 38-43, wherein the second antibody variant is further substituted with glycine at heavy chain position 56 (AHo numbering)

47. The method of any one of claims 33-46, wherein the increased stability of the second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

48. The method of claim 47, wherein the increase in titer is measured by the rate of binding to a protein A coated probe tip using an Octet Forte Bio Instrument.

49. The method of claim 47, wherein the increased yield is measured by protein A or protein G capture.

50. The method of claim 47, wherein the increased purity is measured by SEC of the purified antibodies.

51. The method of claim 47, wherein the reduction in high molecular weight species is measured by SEC and area under the curve for each peak at each molecular weight.

52. The method of claim 47, wherein the increased melting point temperature is measured by DSF or DSC.

53. The method of claim 47, wherein the increased temperature of aggregation is measured by DSF.

54. The method of claim 47, wherein the increased temperature of the onset of melting is measured by DSF.

55. The method of any of claims 33-54, wherein the first antibody variant is a multi-specific antibody.

56. The method of claim 55, wherein the multi-specific antibody is a bispecific antibody or trispecific antibody.

57. The method of any of claims 33-56, wherein the first antibody variant is an antibody fragment that can bind an antigen.

58. The method of claim 57, wherein the antibody fragment is selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

59. The method of any of claims 33-58, wherein the first antibody variant is a human monoclonal antibody or a humanized monoclonal antibody variant.

60. The method of any of claims 33-59, wherein the first antibody variant is an IgG antibody variant.

61. The method of claim 60, wherein the IgG antibody variant is selected from the group consisting of an IgG1, IgG2, IgG3, and IgG4 antibody variant.

62. The method of claim 61, wherein the IgG antibody variant is an IgG1 antibody variant.

63. The method of claim 61, wherein the IgG antibody variant is an IgG2 antibody variant.

64. The method of claim 61, wherein the IgG antibody variant is an IgG3 antibody variant.

65. The method of claim 61, wherein the IgG antibody variant is an IgG4 antibody variant.

66. A method of increasing stability of a first antibody or first antibody variant, comprising
a. identifying the germline original amino acid sequence for the heavy chain of the first antibody or of the antibody portion of the antibody variant;
b. identifying the amino acid residues at heavy chain position 56 (AHo numbering) and heavy chain position 80 (AHo numbering) in the first antibody or of the antibody portion of the antibody variant; and
c. substituting at heavy chain positions 56 and 80 the identified residues from the germline original amino acid sequence in the first antibody or of the antibody portion of the antibody variant, thereby creating a second antibody or second antibody variant,
wherein the second antibody is more stable than the unsubstituted first antibody, or wherein the second antibody variant is more stable than the unsubstituted first antibody variant.

67. The method of claim 66, wherein the increased stability of the second antibody or second antibody variant is demonstrated by at least one selected from the group consisting of an increase in titer during cell culture, increased yield from cell culture, increased purity after purification, a reduction in high molecular weight species, an increased melting point temperature, an increased temperature of aggregation, and an increased temperature of the onset of melting.

68. The method of claim 67, wherein the increase in titer is measured by the rate of binding to a protein A coated probe tip using an Octet Forte Bio Instrument.

69. The method of claim 67, wherein the increased yield is measured by protein A or protein G capture.

70. The method of claim 67, wherein the increased purity is measured by SEC of purified protein.

71. The method of claim 67, wherein the reduction in high molecular weight species is measured by SEC and area under the curve for peaks at each molecular weight

72. The method of claim 67, wherein the increased melting point temperature is measured by DSF or DSC.

73. The method of claim 67, wherein the increased temperature of aggregation is measured by DSF.

74. The method of claim 67, wherein the increased temperature of the onset of melting is measured by DSF.

75. The method of any one of claims 66-74, wherein the first antibody is a monoclonal antibody.

76. The method of claim 75, wherein the first antibody is a human monoclonal antibody or a humanized monoclonal antibody.

77. The method of any one of claims 66-76, wherein the first antibody is an IgG antibody.

78. The method of claim 77, wherein the IgG antibody is selected from the group consisting of an IgG1, IgG2, IgG3, and IgG4 antibody.

79. The method of claim 78, wherein the IgG antibody is an IgG1 antibody.

80. The method of claim 78, wherein the IgG antibody is an IgG2 antibody.

81. The method of claim 78, wherein the IgG antibody is an IgG3 antibody.

82. The method of claim 78, wherein the IgG antibody is an IgG4 antibody.

83. The method of any of claims 66-82, wherein the first antibody variant is a multi-specific antibody.

84. The method of claim 83, wherein the multi-specific antibody is a bispecific antibody or trispecific antibody.

85. The method of any of claims 66-84, wherein the first antibody variant is an antibody fragment that can bind an antigen.

86. The method of claim 85, wherein the antibody fragment is selected from the group consisting of a Fab fragment, a Fab′ fragment, a F′(ab)2 fragment, an Fv fragment, a single chain antibody, diabodies), a biparatopic peptide, a domain antibody (dAb), a CDR-grafted antibody, a single-chain antibody (scFv), a single chain antibody fragment, a chimeric antibody, a diabody, a triabody, a tetrabody, a minibody, a linear antibody; a chelating recombinant antibody, a tribody, a bibody, an intrabody, a nanobody, a small modular immunopharmaceutical (SMIP), an antigen-binding-domain immunoglobulin fusion protein, a single domain antibody, and a VHH containing antibody.

87. The method of any of claims 66-86, wherein the first antibody variant is a human monoclonal antibody or a humanized monoclonal antibody variant.

88. The method of any of claims 66-87, wherein the first antibody variant is an IgG antibody variant.

89. The method of any of claims 66-88, wherein the IgG antibody variant is selected from the group consisting of an IgG1, IgG2, IgG3, and IgG4 antibody variant.

90. The method of claim 89, wherein the IgG antibody variant is an IgG1 antibody variant.

91. The method of claim 89, wherein the IgG antibody variant is an IgG2 antibody variant.

92. The method of claim 89, wherein the IgG antibody variant is an IgG3 antibody variant.

93. The method of claim 89, wherein the IgG antibody variant is an IgG4 antibody variant.

94. The method of any one of claims 1-93, wherein the second antibody or second antibody variant is substituted with any one of the following pairs of residues at positions 56 and 80 of the heavy chain (AHo numbering), respectively: GF, GI, GL, GT, GV, AF, AI, AL, AV, AA, AM, SA, SI, or ST.

95. The method of any of claims 1-94, further comprising formulating the second antibody or second antibody variant into a pharmaceutical composition.

96. An antibody or antibody variant made by the method of any of claims 1-95.

97. A pharmaceutical composition comprising the antibody or antibody variant of claim 96.

98. The method of any of claims 1-93, further comprising formulating the second antibody or second antibody variant into a pharmaceutical composition.

99. An antibody or antibody variant made by the method of any of claims 1-93.

100. A pharmaceutical composition comprising the antibody or antibody variant of claim 99.
